(19) United States
(12) Patent Application Publication
(10) Pub. No.: US 2005/0222247 A1
(43) Pub. Date: Oct. 6, 2005

(54) Chroman derivatives

(75) Inventors: Stephen J. Boyer, Hilden (DE); Kentaro Hashimoto, Wuppertal (DE); Thomas Rolle, Leverkusen (DE); Peter Sandner, Wuppertal (DE); Beatrix Stelte-Ludwig, Wulfrath (DE); Hanna Tinel, Wuppertal (DE); Kerstin Henninger, Wuppertal (DE); Arnel Concepcion, Uji-shi (JP); Osamu Sakurai, Otokuni-gun (JP); Kanako Hirai, Sitama-shi (JP); Tadashi Inoue, Aichi-ken (JP); Yuki Mochizuki, Tokyo-to (JP); Noriko Nunami, Nishinomiya-shi (JP); Masaomi Tajimi, Aichi-ken (JP); Noriyuki Yamamoto, Higashiosaka-shi (JP); Yasuhiro Tsukimi, Amagasaki-shi (JP)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/996,230

(22) Filed: Nov. 23, 2004

(30) Foreign Application Priority Data
Dec. 13, 2003 (EP) 03028781.7

(51) Int. Cl. A61K 31/353; C07D 311/74

(52) U.S. Cl. 514/456; 549/403

(57) ABSTRACT

This invention relates to chroman derivatives and salts thereof which are useful as active ingredients of pharmaceutical preparations. The chroman derivatives of the present invention have an excellent activity as BETA 3 antagonists and are useful for the prophylaxis and treatment of diseases associated with BETA 3 activity, in particular for the treatment of urological disorder or disease, such as detrusor overactivity (overactive bladder), urinary incontinence, neurogenic detrusor oeractivity (detrusor hyperflexia), idiopathic detrusor overactivity (detrusor instability), benign prostatic hyperplasia, and lower urinary tract symptoms; and inflammatory disorders, such as asthma and COPD.

### DETAILED DESCRIPTION OF INVENTION

1. Technical Field

The present invention relates to a novel chroman derivatives which are useful as an active ingredient of pharmaceutical preparations. The chroman derivative of the present invention has beta-3 adrenoreceptor (beta 3) agonistic activity, and can be used for the prophylaxis and treatment of diseases associated with beta 3 activity, in particular for the treatment of urological diseases or disorders, such as detrusor overactivity (overactive bladder), urinary incontinence, neurogenic detrusor overactivity (detrusor hyperflexia), idiopathic detrusor overactivity (detrusor instability), benign prostatic hyperplasia, and lower urinary tract symptoms.

2. Background Art

Adrenoreceptors, or adrenergic receptors, are sites on effecter organs that are innervated by post-ganglionic adrenergic fibers of the sympathetic nervous system, and are classified as either alpha-adrenergic or beta-adrenergic receptors. Alpha-adrenergic receptors respond to norepinephrine and to such blocking agents as phenoxybenzamine and phentolamine, whereas beta-adrenergic receptors respond to epinephrine and to such blocking agents as propranolol.

Beta-adrenergic receptors are sub-classified as beta-1, beta-2, and beta-3 adrenoreceptors. Generally, beta-1 stimulation causes cardiostimulation, whereas beta-2 stimulation causes broncho-dilation and vasodilation. Beta-3 adrenoceptor stimulation causes relaxation of bladder smooth muscle in human (Igawa Y et al. 1998 Acta Physiol Scand 164: 117-118, 1998. Igawa Y et al. Neurourol Urodyn 16: 363-365, 1997. Igawa Y et al. Br J Pharmacol 126: 819-825, 1999.).

Urinary bladder function is controlled by both the parasympathetic and sympathetic nervous systems. Acetylcholine released from parasympathetic nerve causes contraction of bladder via stimulation of muscarinic receptor during urine voiding phase. On the other hand, norepinephrine released from sympathetic nerve causes relaxation of bladder via beta-3 adrenergic receptor during urine storage phase. Therefore, beta-3 adrenoceptor agonist can relax the bladder smooth muscle during urine storage phase, which leads an increase of bladder capacity. Since bladder capacity is decreased in patients with urinary disorders such as urinary incontinence, beta-3 adrenoceptor agonist can be a potential therapeutic benefit for treatment of such urological diseases or disorders.

Further, beta-3 receptors are found on the cell surface of both white and brown adipocytes where their stimulation promotes both lipolysis and energy expenditure. Agonists of beta-3 adrenoreceptors are known to be useful in the treatment of hyperglycemia (diabetes) and obesity in mammals, as well as in the treatment of gastrointestinal disorders and neurogenetic inflammation (U.S. Pat. No. 5,561,142). Additionally, they are known to lower triglyceride and cholesterol levels and to raise high-density lipoprotein levels in mammals (U.S. Pat. No. 5,451,677). Accordingly, they are useful in the treatment of conditions such as hyper triglyceridaemia, hypercholesterolaemia and in lowering high-density lipoprotein levels as well as in the treatment of atherosclerotic and cardiovascular diseases and related conditions. In addition, beta-3 adrenoreceptor agonists may also be useful in treating patients with impaired fasting glucose, impaired glucose tolerance, and type 2 diabetes.

Additionally, it is also believed that the compounds of this invention are effective in the treatment of ocular hypertension and glaucoma, as well as in the treatment of prostate disease and as topical anti-inflammatory agents.

It has now been found that certain novel chroman derivatives are effective as beta-3 agonists and are useful in the treatment of beta-3 mediated conditions.

WO 99/32475 discloses the compounds represented by the general formula: 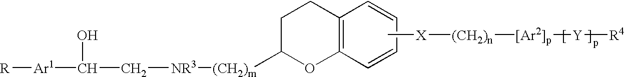
wherein
R is hydrogen, hydroxy, halo etc.; R³ is hydrogen, C₁₋₁₀ alkyl etc.; Ar¹ is Ar¹—O—CH₂, phenyl, or a 5 or 6 membered heterocyclic ring etc.; m is 1, 2, or 3; n is 0, 1, 2, 3, or 4; X is SO₂-piperizinyl, etc.; Ar² is phenyl, or a 5 or 6 membered heterocyclic ring with from 1 to 4 heteroatoms etc.; p is 0 or 1; Y is O—Y C₃-C₈ cycloalkyl etc; and R⁴ is hydrogen, oxo, etc.,
as beta 3 agonists.

WO 99/32476 discloses the compounds represented by the general formula: 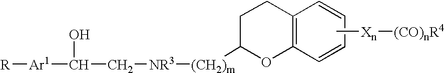
wherein
R is hydrogen, hydroxy, halo etc.; R³ is hydrogen, C₁₋₁₀ alkyl etc.; Ar¹ is phenyl, or a 5 or 6 membered heterocyclic ring etc.; m is 1, 2, or 3; n is 0, 1, or 2; X is C₁₋₆ alkyl optionally substituted with halogen; and R⁴ is hydrogen or C₁₋₆ alkoxy etc.,
as beta 3 agonists.

WO 02/48134 discloses the compounds represented by the general formula: 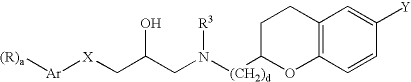
wherein
R is hydrogen, hydroxy, halo etc.; R³ is hydrogen, C₁₋₁₀ alkyl etc.; Ar is phenyl, or a 5 or 6 membered heterocyclic ring etc.; a is 0, 1, 2, 3, 4, or 5; d is 1, 2, or 3; X is O or S(O)_{b}; and Y is halo, phenyl optionally fused to another phenyl ring or to a 5- or 6-membered hyterocycle etc., which is optionally substituted,
as beta 3 agonists.

WO 02/85891 discloses the compounds represented by the general formula: 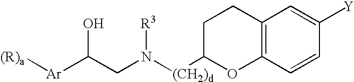
wherein
R is hydroxy, halo etc.; R³ is hydrogen, C₁₋₁₀ alkyl etc.; Ar is phenyl, or a 5 or 6 membered heterocyclic ring etc.; a is 0, 1, 2, 3, 4, or 5; d is 1, 2, or 3; and Y is halo, phenyl optionally fused to another phenyl ring or to a 5- or 6-membered hyterocycle etc., which is optionally substituted,
as beta 3 agonists.

WO 03/24948 discloses the compounds represented by the general formula: 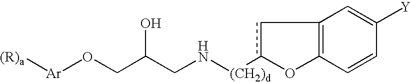
wherein 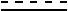
represents a single or double bond; R is hydroxy, halo etc.; Ar is phenyl, or a 5 or 6 membered heterocyclic ring etc.; a is 0, 1, 2, 3, 4, or 5; d is 1, 2, or 3; and Y is C₁₋₁₀ alkyl, halo, phenyl optionally fused to another phenyl ring or to a 5- or 6-membered hyterocycle etc., which is optionally substituted,
as beta 3 agonist.

Yet the development of a compound which has effective and selective beta 3 agonistic activity and can be used for the prophylaxis and treatment of diseases associated with beta 3 activity, in particular for the treatment of urinary incontinence, urge urinary incontinence, overactive bladder as well as inflammatory diseases such as asthma and COPD has been desired.

### SUMMARY OF THE INVENTION

This invention is to provide a chroman derivatives of the formula (I), their tautomeric and stereoisomeric form, and salts thereof: 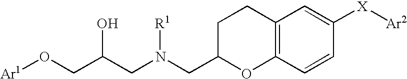
wherein
R¹ represents hydrogen or C₁₋₆ alkyl;
X represents O or NR² (wherein R² represents hydrogen or C₁₋₆ alkyl);
Ar¹ represents phenyl or 5-14 membered heteroaryl containing one, two or three heteroatoms each independently selected from O, S, or N atom
   wherein said phenyl or 5-14 membered heteroaryl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxy-carbonyl), sulfonamide, C₁₋₆ alkanoyl, C₁₋₆ alkanoylamino, carbamoyl, C₁₋₆ alkyl-carbamoyl, cyano, C₁₋₆alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cyclo-alkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, 5-6 membered heteroaryl and heterocyclyl; and
Ar² represents phenyl or 5-6 membered heteroaryl containing one or two heteroatoms each independently selected from O, S, or N atom
   wherein said phenyl or 5-6 membered heteroaryl is substituted by one selected from the group consisting of carboxyl, C₁₋₆ alkoxycarbonyl, hydroxycarbonyl-C₁₋₆ alkyl, hydroxycarbonyl C₁₋₆alkyloxy, carbamoyl, cyano and 5-6 membered unsaturated heterocyclyl,
   and further substituted by one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl, heterocyclyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, 5-6 membered heteroaryl and heterocyclyl.

In another embodiment, the chroman derivatives of formula (I) can be those wherein;
R¹ represents hydrogen;
X represents O;
Ar¹ represents phenyl.
wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₁₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl or 5-6 membered heteroaryl containing one or two heteroatoms each independently selected from O, S, or N atom
   wherein said phenyl or 5-6 membered heteroaryl is substituted by one selected from the group consisting of carboxyl, C₁₋₆ alkoxycarbonyl, hydroxycarbonyl-C₁₋₆ alkyl, hydroxycarbonylC₁₋₆alkyloxy, carbamoyl, tetrazole, 1,2,4-triazole, 5-oxo-1,2,4-oxadiazol, 5-oxo-1,2,4-thiadiazol, 5-thiooxo-1,2,4-oxadiazole, and 1,2,3,5-oxathiadiazole 2-oxide
   and further substituted by one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₀₋₄ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl) amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the chroman derivatives of formula (I) can be those wherein;
R¹ represents hydrogen;
X represents NR² (wherein R² represents hydrogen or C₁₋₆ alkyl);
Ar¹ represents phenyl
   wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl) amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl or 5-6 membered heteroaryl containing one or two heteroatoms each independently selected from O, S, or N atom wherein said phenyl or 5-6 membered heteroaryl is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In a further embodiment, said chroman derivative of the formula (I) can be those wherein;
R¹ represents hydrogen or C₁₋₆ alkyl;
X represents O or NR² (wherein R² represents hydrogen or C₁₋₆ alkyl);
Ar¹ represents phenyl
   wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl
   wherein said phenyl is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxy-carbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

Yet in a further embodiment, said chroman derivative of the formular (I) can be those wherein:
R¹ represents hydrogen;
X represents O or NR² (wherein R² represents hydrogen or C₁₋₆ alkyl);
Ar¹ represents pyridine or pyrimidine
   wherein said pyridine or pyrimidine is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl or 5-6 membered heteroaryl containing one or two heteroatoms each independently selected from O, S, or N atom
   wherein said phenyl or 5-6 membered heteroaryl is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In a further embodiment, said chroman derivative of the formular (I) can be those wherein:
R¹ represents hydrogen or C₁₋₆ alkyl;
X represents O or NR² (wherein R² represents hydrogen or C₁₋₆ alkyl);
Ar¹ represents phenyl
   wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents pyridine or pyrimidine
   wherein said pyridine or pyrimidine is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆alkylamino, di(C₁₋₆alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

Yet in a further embodiment, said chroman derivative of the formular (I), its tautomeric or stereoisomeric form, or a salt thereof, wherein said chroman derivative of the formula (I) is selected from the group consisting of:
4-{[(2R)-2-({[(2S)-2-hydroxy-3-phenoxypropyl]amino}methyl)-3,4-dihydro-2H-chromen-6-yl]oxy}benzoic acid;
3-{[(2R)-2-({[(2S)-2-hydroxy-3-phenoxypropyl]amino}methyl)-3,4-dihydro-2H-chromen-6-yl]oxy}benzoic acid;
4-{[(2R)-2-({[(2S)-2-hydroxy-3-phenoxypropyl]amino}methyl)-3,4-dihydro-2H-chromen-6-yl]oxy}-3-methylbenzoic acid;
methyl 4-{[(2R)-2-({[(2S)-2-hydroxy-3-phenoxypropyl]amino}methyl)-3,4-dihydro-2H-chromen-6-yl]amino}benzoate;
4-{[(2R)-2-({[(2S)-2-hydroxy-3-phenoxypropyl]amino}methyl)-3,4-dihydro-2H-chromen-6-yl]amino}benzoic acid;
3-{[(2R)-2-({[(2S)-2-hydroxy-3-phenoxypropyl]amino}methyl)-3,4-dihydro-2H-chromen-6-yl]oxy}-2-methylbenzoic acid;
methyl 4-[[(2R)-2-({[(2S)-2-hydroxy-3-phenoxypropyl]amino}methyl)-3,4-dihydro-2H-chromen-6-yl](methyl)amino]benzoate;
4-[[(2R)-2-({[(2S)-2-hydroxy-3-phenoxypropyl]amino}methyl)-3,4-dihydro-2H-chromen-6-yl](methyl)amino]benzoic acid;
2-{[(2R)-2-({[(2S)-2-hydroxy-3-phenoxypropyl]amino}methyl)-3,4-dihydro-2H-chromen-6-yl]oxy}benzoic acid;
methyl 3-{[(2R)-2-({[(2S)-2-hydroxy-3-phenoxypropyl]amino}methyl)-3,4-dihydro-2H-chromen-6-yl]amino}benzoate;
3-{[(2R)-2-({[(2S)-2-hydroxy-3-phenoxypropyl]amino}methyl)-3,4-dihydro-2H-chromen-6-yl]amino}benzoic acid;
4-{[(2R)-2-({[(2S)-2-hydroxy-3-phenoxypropyl]amino}methyl)-3,4-dihydro-2H-chromen-6-yl]oxy}-3-methoxybenzoic acid;
3-fluoro-4-{[(2R)-2-({[(2S)-2-hydroxy-3-phenoxypropyl]amino}methyl)-3,4-dihydro-2H-chromen-6-yl]oxy}benzoic acid;
2-fluoro-4-{[(2R)-2-({[(2S)-2-hydroxy-3-phenoxypropyl]amino}methyl)-3,4-dihydro-2H-chromen-6-yl]oxy}benzoic acid;
3-fluoro-4-{[(2R)-2-({[(2S)-2-hydroxy-3-phenoxypropyl]amino}methyl)-3,4-dihydro-2H-chromen-6-yl]amino}benzoic acid;
4-{[(2R)-2-({[(2S)-2-hydroxy-3-phenoxypropyl]amino}methyl)-3,4-dihydro-2H-chromen-6-yl]amino}-3-methylbenzoic acid;
4-{[(2R)-2-({[(2S)-2-hydroxy-3-phenoxypropyl]amino}methyl)-3,4-dihydro-2H-chromen-6-yl]amino}-3-methoxybenzoic acid;
4-{[(2R)-2-({[(2S)-2-hydroxy-3-phenoxypropyl]amino}methyl)-3,4-dihydro-2H-chromen-6-yl]oxy}-3,5-dimethoxybenzoic acid;
3-chloro-4-{[(2R)-2-({[(2S)-2-hydroxy-3-phenoxypropyl]amino}methyl)-3,4-dihydro-2H-chromen-6-yl]oxy}benzoic acid;
3-chloro-4-{[(2R)-2-({[(2S)-2-hydroxy-3-phenoxypropyl]amino}methyl)-3,4-dihydro-2H-chromen-6-yl]oxy}-5-methoxybenzoic acid;
3-{[(2R)-2-({[(2S)-2-hydroxy-3-phenoxypropyl]amino}methyl)-3,4-dihydro-2H-chromen-6-yl]oxy}-4-methylbenzoic acid;
3-{[(2R)-2-({[(2S)-2-hydroxy-3-phenoxypropyl]amino}methyl)-3,4-dihydro-2H-chromen-6-yl]oxy}-5-nitrobenzoic acid;
3-tert-butyl-4-{[(2R)-2-({[(2S)-2-hydroxy-3-phenoxypropyl]amino}methyl)-3,4-dihydro-2H-chromen-6-yl]oxy}benzoic acid;
5-amino-2-{[(2R)-2-({[(2S)-2-hydroxy-3-phenoxypropyl]amino}methyl)-3,4-dihydro-2H-chromen-6-yl]oxy}benzoic acid hydrochloride; and
4-{[(2R)-2-({[(2S)-2-hydroxy-3-phenoxypropyl]amino}methyl)-
3,4-dihydro-2H-chromen-6-yl]oxy}-3-propylbenzoic acid.

The chroman derivatives of formula (I), their tautomeric and stereoisomeric form, and salts thereof surprisingly show excellent beta 3 agonistic activity. They are, therefore suitable especially for the prophylaxis and treatment of diseases associated with beta 3 activity, in particular for the treatment of urological diseases or disorders, such as detrusor overactivity (overactive bladder), urinary incontinence, neurogenic detrusor oeractivity (detrusor hyperflexia), idiopathic detrusor overactivity (detrusor instability), benign prostatic hyperplasia, and lower urinary tract symptoms.

Further, the present invention provides a medicament, which includes one of the compounds, described above and optionally pharmaceutically acceptable excipients.

Alkyl per se and “alk” and “alkyl” in alkenyl, alkynyl, alkoxy, alkanoyl, alkylamino, alkylaminocarbonyl, alkylaminosulfonyl, alkylsulfonylamino, alkoxycarbonyl, alkoxycarbonylamino and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tertbutoxy, n-pentoxy and n-hexoxy.

Alkylamino illustratively and preferably represents an alkylamino radical having one or two (independently selected)alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Cycloalkyl per se and in cycloalkylamino and in cycloalkylcarbonyl represents a cycloalkyl group having generally 3 to 8 and preferably 5 to 7 carbon atoms, illustratively and preferably representing cyclopropyl, cyclobutyl, cyclopentyl, cyclohexyl and cycloheptyl.

Heterocyclyl per se and in heterocyclylcarbonyl represents a mono- or polycyclic, preferably mono- or bicyclic, nonaromatic heterocyclic radical having generally 4 to 10 and preferably 5 to 8 ring atoms and up to 3 and preferably up to 2 hetero atoms and/or hetero groups selected from the group consisting of N, O, S, SO and SO₂. The heterocyclyl radicals can be saturated or partially unsaturated. Preference is given to 5- to 8-membered monocyclic saturated heterocyclyl radicals having up to two hetero atoms selected from the group consisting of O, N and S, such as illustratively and preferably tetrahydrofuran-2-yl, pyrrolidin-2-yl, pyrrolidin-3-yl, pyrrolinyl, piperidinyl, morpholinyl, and perhydroazepinyl.

Aryl per se and in arylamino and in arylcarbonyl represents a mono- to tricyclic aromatic carbocyclic radical having generally 6 to 14 carbon atoms, illustratively and preferably representing phenyl, naphthyl and phenanthrenyl.

Heteroaryl per se and in heteroarylamino and heteroarylcarbonyl represents an aromatic mono-, bi- or tricyclic radical having generally 5 to 14, preferably 5 to 10 and more preferably 5 or 6 ring atoms and up to 5, preferably up to 4 and more preferably up to 3 hetero atoms selected from the group consisting of S, O and N, illustratively and preferably representing thienyl, furyl, pyrrolyl, thiazolyl, oxazolyl, imidazolyl, triazolyl, pyridyl, pyrimidyl, pyridazinyl, indolyl, indazolyl, benzofuranyl, benzothiophenyl, quinolinyl, isoquinolinyl, carbazolyl, carbolinyl, acridinyl and phenazinyl.

### EMBODIMENT OF THE INVENTION

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by any of the Method [A]-[F] below using compound of formula (II) (wherein R¹, and Ar¹ are the same as defined above and L¹ represents a leaving group including, for instance, halogen atom such as chlorine, bromine, fluoride, or iodine atom) as a starting material.
[Method A]

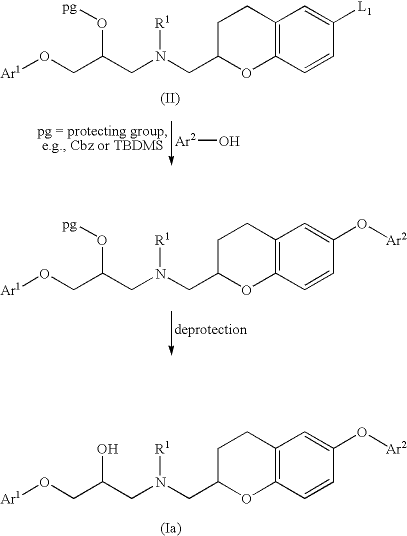

The compound of the formula (Ia) (wherein R¹, Ar¹, and Ar² are the same as defined above) can be prepared by i) reacting the compound of the formula (II) with the compound Ar²—OH (wherein Ar² is the same as defined above) and ii) removing protecting group.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; alcohols such as isopropyl alcohol; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, room temperature to reflux. The reaction may be conducted for, usually, 30 minutes to 48 hours.

The reaction can be advantageously carried out in the presence of a base including, for instance, cesium(ll) carbonate (Cs₂CO₃), sodium carbonate (Na₂CO₃), potassium carbonate (K₂CO₃), organic amines such as pyridine, triethylamine and N,N-diisopropylethylamine, dimethylaniline, diethylaniline, 4-dimethylaminopyridine, and others; catalyst including, for instance, copper catalyst such as copper (I) iodide anhydrate, copper (I) chloride, and copper (I) bromide; and ligand including, for instance, 2,2,6,6-tetramethylheptane-3,5-dione (TMHD).
[Method B]

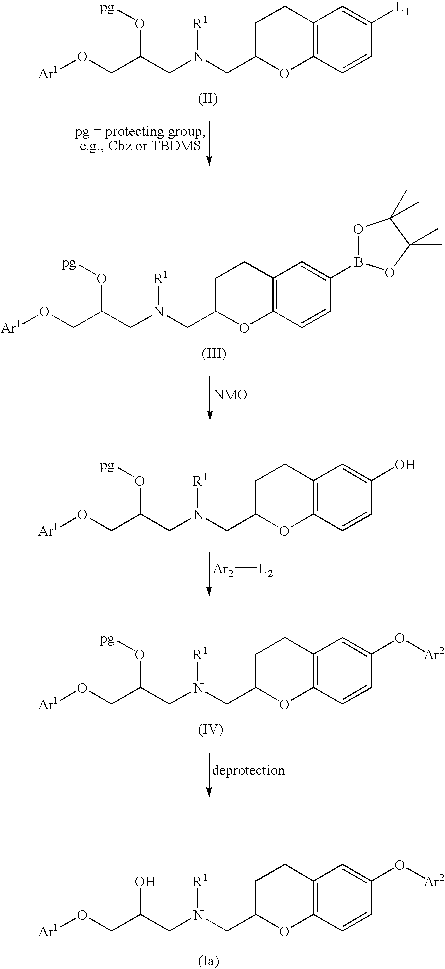

Alternatively, the compound of the formula (Ia) can be prepared by a modified Ullmann condensation reaction. The compound of Formula (II) is first converted to the boronic ester (II) (step 1), which is then converted to the alcohol (IV) by reaction with 4-methylmorpholine N-oxide (NMO) (step 2) and subjected to condensation reaction with a Ar²—L₂ (wherein Ar² is the same as defined above and L₂ is a leaving group including, for instance, halogen atom such as chlorine, bromine, floride or iodine atom) to provide Formula (IV) compound (step 3). Then a protecting group is removed.

In the all steps, the reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, rt to reflux. The reaction may be conducted for, usually, 30 minutes to 48 hours.

The step 1 can be advantageously carried out in the presence of a base including, for instance, sodium carbonate (Na₂CO₃), cesium(ll) carbonate (Cs₂CO₃), potassium carbonate (K₂CO₃), organic amines such as pyridine, triethylamine and N,N-diisopropylethylamine, dimethylaniline, diethylaniline, 4-dimethylaminopyridine, and others; catalyst including, for instance, palladium catalyst; and pinnacol borane or bispinacol borane.

In the step 3, the reaction can be advantageously carried out in the presence of a base including, for instance, sodium carbonate (Na₂CO₃), cesium(ll) carbonate (Cs₂CO₃), and potassium carbonate, organic amines such as pyridine, triethylamine and N,N-diisopropylethylamine, dimethylaniline, diethylaniline, 4-dimethylaminopyridine, and others; copper catalyst including, for instance, copper (I) iodide anhydrate, copper (I) chloride, and copper (I) bromide.
[Method C]

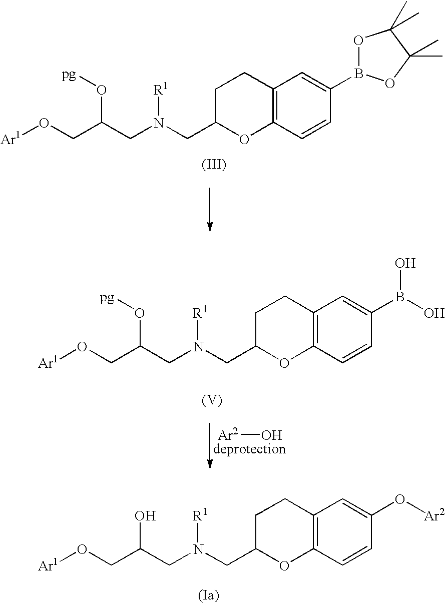

Further, the compound of the formula (Ia) can be prepared by i) hydrolyzing the compound of the formula (III) to make boronic acid compound (V) (step 1) and ii) reacting the compound (V) with the compound Ar²—OH (wherein Ar² is the same as defined above) (step 2).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitrites such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, room temperature to reflux. The reaction may be conducted for, usually, 30 minutes to 24 hours and preferably 1 to 10 hours.

In the step 1 the reaction can be advantageously carried out in the presence of a base including, for instance, organic amines such as pyridine, triethylamine and N,N-diisopropylethylamine, dimethylaniline, diethylaniline, 4-dimethylaminopyridine, and others such as ammonium acetate; and reacting agent (oxidant) like sodium periodate.

In the step 2 the reaction can be advantageously carried out in the presence of a base including, for instance, organic amines such as pyridine, triethylamine and N,N-diisopropylethylamine, triethylamine, dimethylaniline, diethylaniline, 4-dimethylaminopyridine, and others; catalyst including, for instance, copper catalyst including, for instance, cupric acetate.
[Method D]

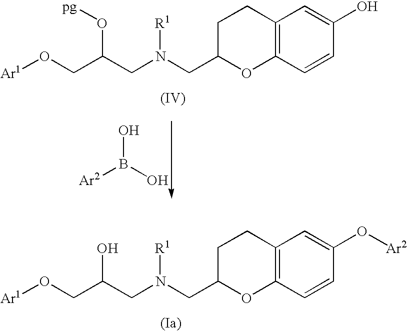

Alternatively, the compound of the formula (Ia) can be prepared by reacting the compound (TV) with the compound Ar²—B(OH)₂ (wherein Ar² is the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, room temperature to reflux. The reaction may be conducted for, usually, 30 minutes to 24 hours and preferably 1 to 10 hours.

The reaction can be advantageously carried out in the presence of a base including, for instance, cesium(ll) carbonate (CS₂CO₃), sodium carbonate (Na₂CO₃), potassium carbonate (K₂CO₃), organic amines such as pyridine, triethylamine and N,N-diisopropylethylamine, triethylamine, dimethylaniline, diethylaniline, 4-dimethylaminopyridine; and catalyst including, for instance, copper catalyst including, for instance, cupric acetate.
[Method E]

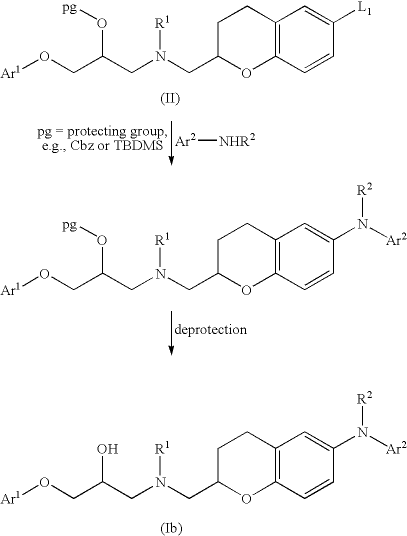

The compound of the formula (Ib) (wherein R¹, R², Ar¹, and Ar² are the same as defined above) can be prepared by i) reacting the compound of the formula (II) with the compound Ar²—NHR² (wherein Ar² and R² are the same as defined above) and ii) removing protecting group.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, room temperature to reflux. The reaction may be conducted for, usually, 30 minutes to 48 hours.

The reaction can be advantageously carried out in the presence of a base including, for instance, cesium(ll) carbonate (Cs₂CO₃), sodium carbonate (Na₂CO₃), potassium carbonate (K₂CO₃), organic amines such as pyridine, triethylamine and N,N-diisopropylethylamine, dimethylaniline, diethylaniline, 4-dimethylaminopyridine, and others; catalyst including, for instance,; palladium catalyst such as Pd(OAc)₂ and Pd₂(dba)₃; and ligand including, for instance, biaryl dialkyl-phosphine and 2,2,6,6-tetramethylheptane-3,5-dione (TMHD).
[Method F]

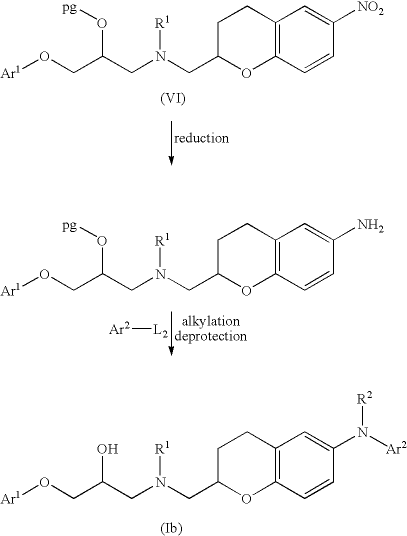

Alternatively, the compound of the formula (Ib) can be prepared from the nitro compound of Formula (VI) by reduction to the compound to formula (VII) (step 1) followed by alkylation and deprotection (step 2).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, room temperature to reflux. The reaction may be conducted for, usually, 30 minutes to 48 hours.

In the step 1, the reaction can be advantageously carried out in the presence of a reducing agent. In the step 2, the reaction can be advantageously carried out in the presence of base including, for instance, cesium(ll) carbonate (Cs₂CO₃), sodium carbonate (Na₂CO₃), potassium carbonate (K₂CO₃), organic amines such as pyridine, triethylamine and N,N-diisopropylethylamine, di-methylaniline, diethylaniline, 4-dimethylaminopyridine, and others; catalyst including, for instance,; palladium catalyst such as Pd(OAc)₂ and Pd₂(dba)₃; and lignad including, for instance, biaryl dialkylphosphine and 2,2,6,6-tetramethylheptane-3,5-dione (TMHD).

### Preparation of Starting Materials

The compound of the formula (II) that can be used as a starting material of the compound of the formula (I) can be, but not limited to be, prepared by any of the Method [a]-[c] below.
[Method a]

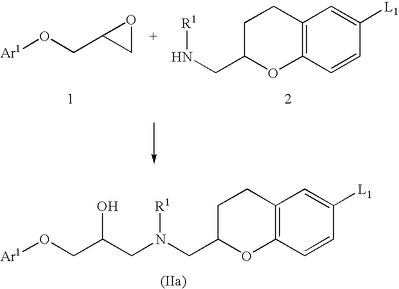

The compound of the formula (IIa) can be prepared by reacting the compound of the formula 1 with the compound of formula 2.

The reaction may be carried out in an solvent including, for instance, dimethyl sulfoxide, dimethyl formamide, acetonitrile, or in an alcohol such as ethanlo, isopropanol, or propanol; halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene and others. Optionally, two or more of the solvents selected from the listed above can be mixed and used.

The reaction temperature can be optionally set depending on the compounds to be reacted. The reaction temperature is usually, but not limited to, about −0° C. to reflux. The reaction may be conducted for, usually, 30 minutes to 24 hours and preferably 1 to 10 hours.

Compounds (IIa) in which R¹ is other than hydrogen may be prepared by reaction of compound (IIa) in which R¹ is hydrogen by selective N-alkylation or N-acylation reactions with known compounds of formula R¹-halo.

The epoxide compounds 1 are commercially available or may be prepared according to one of the many procedures described in the literature known to those skilled in the art. The compound 2 can be prepared standard methods, for example, but not limited to involving conversion of a carboxylic acid to an amide and reduction.
[Method b]

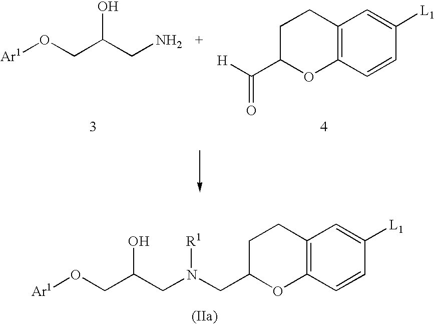

Alternatively, the compound of the formula (IIa) can be prepared by reductive amination with the reaction of an aldehyde of formula 4 and an amino alcohol of formula 3.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 50° C. The reaction may be conducted for, usually, 30 minutes to 24 hours and preferably 1 to 10 hours.

The reaction can be advantageously carried out in the presence of a base including, for instance, cesium(ll) carbonate (Cs₂CO₃), organic amines such as pyridine, triethylamine and N,N-diisopropylethylamine, dimethylaniline, diethylaniline, 4-dimethylaminopyridine, and others.

The amino alcohols 3 are either commercially available or may be prepared by ring opening of the epoxides 1 with a nitrogen nucleophile, such as dibenylamine or phthalimide, in the presence of base.

The compound 4 can be prepared by corresponding carboxylic acid of formula 5 by reduction with borane followed by an axidation.
[Method c]

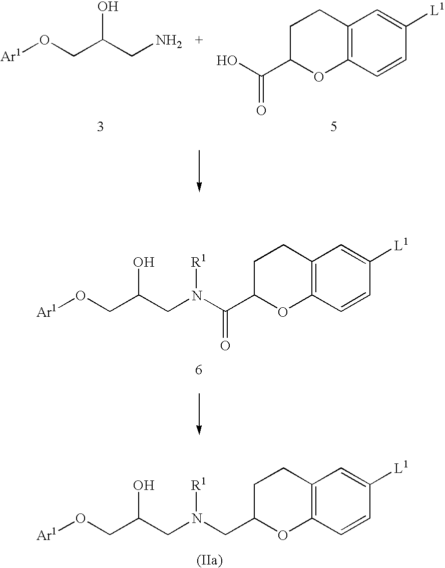

A third general route to Formula (IIa) is reacting an amino alcohol 3 and a carboxylic acid 5 to produce the amide compounds 6 and then reducing the amides 6.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitrites such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 50° C. The reaction may be conducted for, usually, 30 minutes to 24 hours and preferably 1 to 10 hours.

The reaction of reduction can be advantageously carried out in the presence of a base including, for instance, cesium(ll) carbonate (Cs₂CO₃), sodium carbonate (Na₂CO₃), potassium carbonate (K₂CO₃), organic amines such as pyridine, triethylamine and N,N-diisopropylethylamine, dimethylaniline, diethylaniline, 4-dimethylaminopyridine, and others; and reagent like borane dimethylsulfide complex.

The compound 5 can be prepared from the known unsubstituted chroman carboxylic acid by various aromatic substitution reactions at the 6-position of the chroman ring and further elaboration of these products.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, respectively. Acids to form acid addition salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic acid, p-bromophenylsulfonic acid, carbonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include sodium hydroxide, potassium hydroxide, potassium carbonate, sodium carbonate, sodium bicarbonate, potassium bicarbonate, calcium hydroxide, calcium carbonate, and the like.

The compound of the present invention or a salt thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients therefore. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and the like; together with, optionally, disintegrating agents, such as, without limitation, maize, starch, methyl cellulose, agar bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carriers, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described as a form of examples, but they should by no means be construed as defining the metes and bounds of the present invention.

In the examples below, all quantitative data, if not stated otherwise, relate to percentages by weight.

The effect of the present compounds can be examined by the following assays and pharmacological tests.

Measurement of cAMP Production in SK-N-MC Cells (Assay 1—Method A)

Human neuroblastoma cell line, SK-N-MC, which endogenously express β1- and β3-adrenoceptors was utilized. In the presence of 1 μM of 1-adrenoceptor selective antagonist, CGP20712A, the effects of the compounds on cAMP levels were examined. SK-N-MC cells were suspended in Hank's balanced salt solution containing 20 mM Hepes, 0.1% BSA, 1 mM L-ascorbic acid sodium salt, 250 nM IBMX, and 1 μM CGP20712A (pH 7.4). After incubating at 37° C. for 30 min, the compound of the present invention was added and cells were further incubated for 30 min. Total cAMP in the well was measured by cAMP ELISA kit (Tropix, Bedford, Mass.). Effect of the compound on the cAMP level was determined at 6 different concentrations from 0.1 nM to 10 μM. The concentration to induce 50% of maximum response, 50% effective concentration (EC₅₀), was calculated. In addition, intrinsic activity (IA) was determined as a maximum response induced by each compound, and IA was expressed as relative value compared with a response induced by 10 μM isoproterenol (i.e. cAMP level increased by 10 μM isoproterenol was taken as 100%).

Measurement of cAMP production in SK-N-MC Cells (Assay 1—Method B)

Human neuroblastoma cell line, SK-N-MC, which endogenously express β3- and β3-adrenoceptors were utilized. In the presence or absence of 1 μM of β1-adrenoceptor selective antagonist, Atenolol, the effects of the compounds on cAMP levels were examined. SK-N-MC cells were suspended in Hank's balanced salt solution containing 20 mM Hepes, 0.1% BSA, 1 mM L-ascorbic acid sodium salt, 250 nM IBMX, and 1 μM Atenolol (pH 7.4). After incubating at 37° C. for 1 h, the compound of the present invention was added and cells were further incubated for 30 min. Total cAMP in the well was measured by cAMP ELISA kit (Tropix, Bedford, Mass.). Effect of the compound on the cAMP level was determined at 8 different concentrations from 1 pM to 10 μM. The concentration to induce 50% of maximum response, 50% effective concentration (EC₅₀), was calculated.

TABLE 1
Beta-3 Agonist Activity

| Example | Assay | Beta-3 EC₅₀ |
|---|---|---|
| No. | method | [nM] |
| 1 | A | 26 |
| 2 | A | 14 |
| 11 | B | 186 |
| 12 | A | 270 |
| 29 | A | 160 |
| 37 | A | 230 |
| 44 | B | 23 |

Measurement of Agonistic Activity for Human β1-Adrenoceptor Or Human β2-Adrenoceptor (Assay 2—Method A)

The agonistic activity of the compound to human O₂-adrenoceptor was examined by measurement of cAMP levels in Chinese hamster ovary (CHO) cells, in which recombinant human O₂-adrenoceptor was expressed (hβ2-CHO cells). The hβ2-CHO cells were suspended in Hank's balanced salt solution containing 20 mM Hepes, 0.1% BSA, 1 mM L-ascorbic acid sodium salt, and 250 nM IBMX (pH 7.4). After incubating at 37° C. for 30 min, the compound of the present invention was added and cells were further incubated for 30 min. Total cAMP in the well was measured by cAMP ELISA kit (Tropix, Bedford, Mass.). The effect of the compound on the cAMP level was determined at 6 different concentrations from 0.1 nM to 10 μM. The concentration to induce 50% of maximum response, 50% effective concentration (EC₅₀), was calculated. In addition, intrinsic activity (IA) was determined as a maximum response induced by each compound, and IA was expressed as relative value compared with a response induced by 10 μM isoproterenol (i.e. cAMP level increased by 10 μM isoproterenol was taken as 100%). Experiments with the same methods were performed in CHO cells expressing recombinant human β1-adrenoceptor to examine the effects of the compounds on human β1-adrenoceptor.

Measurement of Agonistic Activity for Human β1-Adrenoceptor or Human β2-Adrenoceptor (Assay 2—Method B)

The agonistic activity of the compound to human β2-adrenoceptor was examined by measurement of calcium influx in Chinese hamster ovary (CHO) cells, in which recombinant human β2-adrenoceptor was expressed (hβ2-CHO cells). The cells were cultivated in DMEM F12 medium. Prior to measurement, the cells were loaded with Coelenterazine (1:2000) in Ca-Tyrode. The Ca-influx was directly measured for 45 sec (Hamamatsu FluoroBox fluorescence detector). The effect of the compound on calcium influx was determined at 8 different concentrations from 1 pM to 10 μM. The concentration to induce 50% of maximum response, 50% effective concentration (EC₅₀), was calculated. Experiments with the same methods were performed in CHO cells expressing recombinant human β1-adrenoceptor to examine the effects of the compounds on human β1-adrenoceptor.

TABLE 2
Beta-1, Beta-2 Agonist Activity

| Example | Assay | Beta-1 EC₅₀ | Beta-2 EC₅₀ |
|---|---|---|---|
| No. | method | [μM] | [μM] |
| 1 | A | >10 | >10 |
| 23 | A | | >10 |
| 24 | A | >10 | >10 |
| 44 | B | >10 | >10 |
| 54 | B | 8 | >10 |

Measurement of Antagonistic Activity for Human 01-Adrenoceptor or Human β1-adrenoceptor (Assay 3)

The antagonistic activity of the compound to human β2-adrenoceptor was examined by measurement of cAMP levels in the hβ2-CHO cells stimulated by isoproterenol. The hβ2-CHO cells were suspended in Hank's balanced salt solution containing 20 mM Hepes, 0.1% BSA, 1 mM L-ascorbic acid sodium salt, and 250 nM IBMX (pH 7.4). The cells were stimulated by non-selective β-adrenoceptor agonist isoproterenol at 100 nM to increase cAMP levels. After incubating at 37° C. for 30 min, the compound of the present invention was added and cells were further incubated for 30 min. Total cAMP in the well was measured by cAMP ELISA kit (Tropix, Bedford, Mass.). Inhibitory effect of the compound on the isoproterenol-induced cAMP production was determined at 6 different concentrations from 0.1 nM to 10 μM. The concentration to induce 50% of inhibitory response, 50% inhibitory concentration (IC₅₀), was calculated. Experiments with the same methods were performed in CHO cells expressing recombinant human β1-adrenoceptor to examine the effects of the compounds on human β1-adrenoceptor.

TABLE 3
Beta-1, Beta-2 Antagonist Activity

| Example | Beta-1 IC₅₀ | Beta-2 IC₅₀ |
|---|---|---|
| No. | [μM] | [μM] |
| 1 | >10 | 0.93 |
| 24 | 6.8 | 6.3 |

Organ Bath Assay to Measure Bladder Contraction (Assay 4)

Male Wistar rats (10 week old) were anesthetized with ether and sacrificed by dislocating the necks. The whole urinary bladder was excised and placed in oxygenated Modified Krebs-Henseleit solution (pH 7.4) of the following composition (112 mM NaCl, 5.9 mM KCl, 1.2 mM MgCl₂, 1.2 mM NaH₂PO₄, 2 mM CaCl₂, 2.5 mM NaHCO₃, 12 mM glucose). Contractile responses of the urinary bladder were studied as described previously [Takeda H et al., J. Pharmacol. Exp. Ther. 126: 939-945, 2000]. Isometric tension was recorded under a load of 1 g using longitudinal strips of rat detrusor muscle. Bladder strips were equilibrated for 60 min before each stimulation. Contractile response to 80 mM KCl was determined at 15 min intervals until reproducible responses were obtained. The effects of the compounds on muscle tension were investigated by incubating the strips with β3-adrenoceptor agonist for 30 min.

Measurement of Bladder Pressure in Anesthetized Rats (Assay 5)

Effect of a compound on bladder pressure in rats was studied as described previously [Takeda H et al., J. Pharmacol. Exp. Ther. 293: 939-945, 2000].

Male rats, weighing from 300 to 350 g, were anesthetized with urethane (1.2 g/kg i.p.). Through a midline abdominal incision, the pelvic viscera were exposed, and the ureter on each side was ligated and cut proximal to the ligature so as to allow urine to drain into cotton wads. After the urethra had been ligated, a polyethylene catheter (PE-50; Nihon Becton Dickinson, Tokyo, Japan) was inserted into the urinary bladder via the top of the bladder dome and connected through a three-way connector to a pressure transducer (Viggo-Spectramed Pte Ltd, DT-XXAD) and a syringe filled with saline. The initial bladder pressure was adjusted to 6 cm H₂O by instillation of saline in 0.05 ml increments. Effect of the compound on bladder pressure was quantified by expressing postadministration value as a percentage of the value before drug administration. A venous catheter (PE-50; Nihon Becton Dickinson) was inserted into the left femoral vein for injection of the compound.

Cystometry in Anesthetized Rats (Assay 6)

Effect of a compound on cystometric parameters in rats were studied as described previously [Takeda H et al., J. Pharmacol. Exp. Ther. 293: 939-945, 2000].

Female rats, weighing from 200 to 230 g, were anesthetized with urethane (1.2 g/kg i.p.). Through a midline abdominal incision, the ureter on each side was ligated and cut proximal to the ligature. A polyethylene catheter (PE-50) was inserted into the urinary bladder and connected through a three-way connector to 1) a pressure transducer (Viggo-Spectramed Pte Ltd, DT-XXAD) for measurement of bladder pressure, and 2) a syringe infusion pump (TERUMO) for continuous infusion of saline into the bladder. During cystometry, saline was infused at a rate of 2.4 ml/h. Bladder pressure was recorded continuously on a PowerLab system (BioResearch Center). The following cystometric parameters were obtained: micturition interval and micturition pressure (maximum bladder pressure during micturition). Two reproducible micturition cycles were recorded before drug administration and used to provide a baseline value to be compared with the first two micturition cycles just after drug administration. Relative values for the various cystometric parameters were calculated as follows: (mean value from two micturition cycles just after drug administration)/(mean value from two micturition cycles just before drug administration). A venous catheter was inserted into the left femoral vein for drug injection.

Liquid Chromatography—Mass spectroscopy (LC-MS)— Method 1:

Micromass Platform LC with Shimadzu Phenomenex ODS column (4.6 mm×30 mm) flushing a mixture of acetonitrile-water (9:1 to 1:9) at a flow rate of 1 ml/min. Mass spectra were obtained either by electrospray ionization (ESI): Perkin Elmer/SCIEX API 150MCA, or by direct chemical ionization (DCI): Finnigan MAT 95.

Liquid Chromatography—Mass spectroscopy (LC-MS)— Method 2:

Instrument MS: Micromass ZQ; Instrument HPLC: Waters Alliance 2795; Column: Phenomenex Synergi 2μ Hydro-RP Mercury 20 mm×4 mm; Eluant A: 1 1 water+0.5 ml 50% formic acid, Eluant B: 1 1 acetonitrile+0.5 ml 50% formic acid; Gradient: 0.0 min 90% A→2.5 min 30% A→3.0 min 5% A →4.5 min 5% A; Flow rate: 0.0 min 1 ml/min→>2.5 min/3.0 min/4.5 min 2 ml/min; Oven: 50° C.; UV detection: 210 nm.

Liquid Chromatography—Mass spectroscopy (LC-MS)— Method 3:

Instrument MS: Micromass ZQ; Instrument HPLC: HP 1100 Series; UV DAD; Column: Phenomenex Synergi 2μ Hydro-RP Mercury 20 mm×4 mm; Eluant A: 1 1 water+0.5 ml 50% formic acid, Eluant B: 1 1 acetonitrile+0.5 ml 50% formic acid; Gradient: 0.0 min 90% A→2.5 min 30% A→3.0 min 5% A→4.5 min 5% A; Flow rate: 0.0 min 1 ml/min→2.5 min/3.0 min/4.5 min 2 ml/min; Oven: 50° C.; UV detection: 210 nm.

Liquid Chromatography—Mass spectroscopy (LC-MS)—Method 4

Instrument: Micromass Quattro LCZ with HPLC Agilent Series 1100; Column: Phenomenex Synergi 2μ, Hydro-RP Mercury 20 mm×4 mm; Eluant A: 1 1 water+0.5 ml 50% formic acid, Eluant B: 1 1 acetonitrile+0.5 ml 50% formic acid; Gradient: 0.0 min 90% A→2.5 min 30% A→3.0 min 5% A→4.5 min 5% A; Flow rate: 0.0 min 1 ml/min→2.5 min/3.0 min/4.5 min 2 ml/min; Oven: 50° C.; UV detection: 208-400 nm.

Melting point determinations:

Finnigan M AT95 melting points are uncorrected.

All starting materials are commercially available or can be prepared using methods cited in the literature.

### Preparation Of Intermediates

### Preparation 1

### Preparation of (2R)-6-iodo-3,4-dihydro-2H-chromene-2-carboxylic Acid

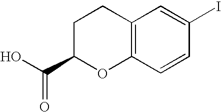

(2R)-3,4-Dihydro-2H-chromene-2-carboxylic acid (prepared as described in WO 99/32476) (13.33 g, 74.82 mmol), benzyltrimethyl-ammonium dichloroiodate (25.0 g, 71.83 mmol) and zinc chloride (12.65 g, 92.78 mmol) were stirred in glacial acetic acid (250 mL) under argon at room temperature for 18 hours. The solid was removed by vacuum filtration and then washed with acetic acid (50 mL). The filtrate was concentrated in vacuo to obtain a solid which was slurried in water (300 mL). The crude product was obtained as a pink solid after vacuum filtration and dried (18.7 g, 82.2%): ¹H NMR (DMSO-d₆) δ 1.95-2.10 (m, 1H), 2.60 (m, 1H), 2.70-2.80 (m, 1H), 4.79 (dd, J=6.0, 3.9 Hz, 1H), 6.63 (d, J=8.4 Hz, 1H), 7.36 (dd, J=8.1, 1.8 Hz, 1H), 7.38 (d, J=1.8 Hz, 1H); CI-MS m/z 305 (M+H⁺). The crude product was used for the next step directly.

### Preparation 2

### Preparation of (2S)-2-(phenoxymethyl)oxirane

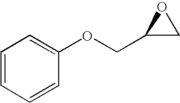

A solution of phenol (17.57 g, 76.97 mmol) in dry DMF (200 mL) was added slowly to a suspension of sodium hydride (60% in mineral oil, 4.0 g, 100.06 mmol) in DMF at 0° C. and stirred at the same temperature for 30 minutes. Then, (2S)-(+)-glycidyl tosylate (17.57 g, 76.97 mmol) was added slowly. The resulting mixture was stirred at room temperature overnight and quenched with saturated ammonium chloride solution. The two-phase mixture was diluted with water and extracted with diethyl ether. The combined organic extracts were washed with saturated NaHCO₃, brine, dried over anhydrous sodium sulfate, concentrated and purified by medium pressure column chromatography (eluant: hexanes/EtOAc 13:1). The product was obtained as a colorless oil in 73% yield.

### Preparation 3

### Preparation of (2S)-1-(dibenzylamino)-3-phenoxy-2-propanol

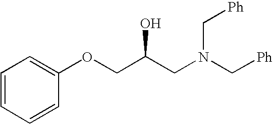

A reaction mixture containing (2S)-2-(phenoxymethyl)oxirane (Preparation 2, 8.44 g, 65.20 mmol) and dibenzylamine (12.20 g, 61.82 mmol, 1.1 eq.) in MeOH (300 mL) was heated at reflux overnight. The resulting solution was concentrated in vacuo and the crude product was purified by medium pressure column chromatography (Biotage 40S normal phase silica gel column, eluant: hexanes/EtOAc 10:1). The product was obtained as a colorless oil in 99% yield. LC-MS, Method 1: M+H⁺=348.3, retention time=2.22 min; R_{f}=0.42 (hexanes/EtOAc 6:1).

### Preparation 4

### Preparation of (2S)-1-amino-3-phenoxy-2-propanol

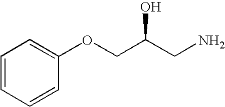

A suspension of (2S)-1-(dibenzylamino)-3-phenoxy-2-propanol (Preparation 3, 19.07 g, 54.88 mmol), palladium hydroxide (20 wt.-% Pd (dry basis) on carbon, Pearlman's catalyst, 0.23 g/mmol) in MeOH/EtOAc (150 mL/150 mL) was stirred under hydrogen atmosphere (H₂ balloon) for 5 hours. The resulting mixture was filtered through a Celite® pad and the pad was washed with MeOH. The filtrate was concentrated in vacuo to afford a yellow solid that was washed with diethyl ether. The resulting residue was purified by medium pressure column chromatography (Biotage 40S normal phase silica gel column, eluant: EtOAc/2 M NH₃ in MeOH 95:5). The product was obtained in 98.1% yield (9.00 g). LC-MS, Method 1: M+H⁺=168.1, retention time=0.76 min; R_{f}=0.12 (EtOAc/2 M NH₃ in MeOH 5:1).

### Preparation 5

### Preparation of (2R)-N-[(2S)-2-hydroxy-3-phenoxypropyl]-6-iodo-3,4-dihydro-2H-chromene-2-carboxamide

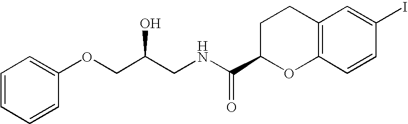

A solution containing (2S)-1-amino-3-phenoxy-2-propanol (Preparation 4, 8.86 g, 52.99 mmol), (2R)-6-iodo-3,4-dihydro-2H-chromene-2-carboxylic acid (Preparation 1, 16.11 g, 52.99 mmol), 1-(3-dimethylaminopropyl)-3-ethylcarbodiimide hydrochloride (20.32 g, 105.98 mmol), 1-hydroxy-benzotriazole (14.32 g, 105.98 mmol), and triethylamine (14.77 mL, 105.98 mmol) in DMF (300 mL) was stirred at room temperature for 5 hours. To the resulting solution was added water and the two-phase mixture was extracted with EtOAc. The organic extracts were washed with water and brine, dried over anhydrous sodium sulfate, concentrated and purified by medium pressure column chromatography (silica gel column, eluant: hexanes/EtOAc 2:1). The product was obtained as a white solid in 64.6% yield (15.52 g). LC-MS, Method 1: M+H⁺=454.1, retention time=3.03 min.

### Preparation 6

### Preparation of (2S)-1-({[(2R)-6-iodo-3,4-dihydro-2H-chromen-2-yl]methyl}amino)-3-phenoxy-2-propanol

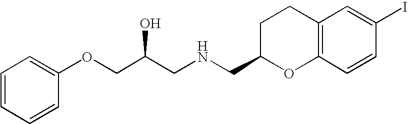

To a solution containing (2R)-N-[(2S)-2-hydroxy-3-phenoxypropyl]-6-iodo-3,4-dihydro-2H-chromene-2-carboxamide (Preparation 5, 15.52 g, 34.24 mmol) in THF (500 mL) at room temperature was slowly added borane-methyl sulfide complex (2 M in THF, 85.60 mL, 171.20 mmol). After completion of addition, the solution was heated to reflux, maintained at that temperature for 2 hours, and then cooled to room temperature. The resulting solution was quenched with EtOH (10 mL) dropwise, then with 2 M HCl (40 mL) slowly. The resulting mixture was heated at reflux for 1 hour and was then allowed to cool to room temperature. This solution was made basic with 1 N NaOH and extracted with ethyl acetate. The organic extract was washed with brine, dried over anhydrous sodium sulfate, and concentrated in vacuo. The resulting residue was dissolved in MeOH and EtOAc and filtered. The filtrate was concentrated and dried in vacuo to afford the product as a white solid in quantitative yield (15.46 g). LC-MS, Method 1: M+H⁺=440.2, retention time=2.24 min.

### Preparation 7

### Preparation of tert-butyl(2S)-2-hydroxy-3-phenoxypropyl{[(2R)-6-iodo-3,4-dihydro-2H-chromen-2-yl]methyl}carbamate

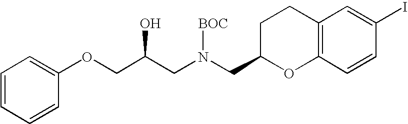

A reaction mixture containing (2S)-1-({[(2R)-6-iodo-3,4-dihydro-2H-chromen-2-yl]methyl}-amino)-3-phenoxy-2-propanol (Preparation 6, 15.47 g, 35.22 mmol) and di-tert-butyl dicarbonate (8.07 g, 36.98 mmol) in THF (350 mL) was stirred at room temperature for 5 hours. To this solution was added water and the resulting two-phase mixture was extracted with ethyl acetate. The organic extract was washed with brine, dried over anhydrous sodium sulfate, concentrated and purified by medium pressure column chromatography (silica gel column, eluant: hexanes/EtOAc 3.5:1). The product was obtained as a colorless oil in quantitative yield (19.00 g). LC-MS, Method 1: M+H+=539.9, retention time=3.99 min.

### Preparation 8

### Preparation of tert-butyl(2S)-2-{[tert-butyl(dimethyl)silyl]oxy}-3-phenoxypropyl{[(2R)-6-iodo-3,4-dihydro-2H-chromen-2-yl]methyl}carbamate

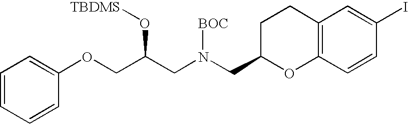

A reaction mixture containing tert-butyl(2S)-2-hydroxy-3-phenoxypropyl {[(2R)-6-iodo-3,4-dihydro-2H-chromen-2-yl]methyl}carbamate (Preparation 7, 19.00 g, 35.22 mmol), tert-butyldimethylsilyl chloride (6.90 g, 45.79 mmol), and imidazole (6.23 g. 91.58 mmol) in anhydrous DMF (70 mL) was stirred at room temperature overnight. The resulting mixture was then poured into water, and extracted with diethyl ether. The organic extract was washed with water and brine, dried over anhydrous sodium sulfate, concentrated, and purified by medium pressure column chromatography (silica gel column, eluant: hexanes/EtOAc 100:5). The product was obtained as a colorless oil in quantitative yield (23.00 g). LC-MS, Method 1: M+H⁺=654.0, retention time=5.29 min.

### Preparation 9

### Preparation of tert-butyl((2S)-2-{[tert-butyl(dimethyl)silyl]oxy}-3-phenoxypropyl){[(2R)-6-(4,4,5,5-tetramethyl-1,3,2-dioxaborolan-2-yl)-3,4-dihydro-2H-chromen-2-yl]methyl}carbamate

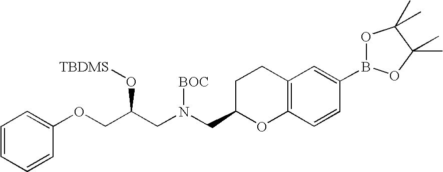

Argon was bubbled for 30 min through a reaction mixture containing tert-butyl(2S)-2-{[tert-butyl(dimethyl)silyl]oxy}-3-phenoxypropyl {[(2R)-6-iodo-3,4-dihydro-2H-chromen-2-yl]methyl}-carbamate (Preparation 8, 3.95 g, 6.04 mmol), bis(pinacolato)borane (1.68 g, 6.64 mmol), and potassium acetate (1.77 g. 18.1 mmol) in anhydrous DMF (30 mL). To the degassed mixture was added Pd(OAc)₂ (0.135 g, 0.604 mmol), and the mixture was stirred at 85° C. overnight. The resulting mixture was quenched with water, and extracted with ethyl acetate. The organic extracts were washed with brine, dried over sodium sulfate, concentrated, and purified by column chromatography (eluant: cyclohexanes/EtOAc 8:1). The product was obtained as a colorless oil in 83% yield (3.47 g). LC-MS, Method 2: M+H⁺=654.4, retention time=3.88 min.

### Preparation 10

### Preparation of tert-butyl((2S)-2-{[tert-butyl(dimethyl)silyl]oxy}-3-phenoxypropyl){[(2R)-6-hydroxy-3,4-dihydro-2H-chromen-2-yl]methyl}carbamate

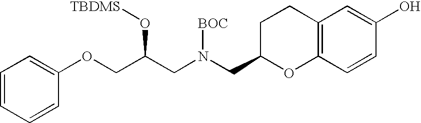

A reaction mixture containing tert-butyl((2S)-2-{[tert-butyl(dimethyl)silyl]oxy}-3-phenoxy-propyl){[(2R)-6-(4,4,5,5-tetramethyl-1,3,2-dioxaborolan-2-yl)-3,4-dihydro-2H-chromen-2-yl]-methyl}carbamate (Preparation 9, 3.47 g, 5.30 mmol) and 4-methylmorpholine 4-oxide (1.55 g, 13.2 mmol) in anhydrous THF (35 mL) was stirred at reflux for overnight. After cooling to room temperature, the resulting mixture was partitioned between water and ethyl acetate. The organic layer was washed with water and brine, and dried over sodium sulfate. After removal of the volatiles in vacuo, the crude product was further purified by column chromatography (eluant: cyclohexanes/EtOAc, gradient 8:1-3:1) giving the product as a colorless oil in 83% yield (2.390 g). LC-MS, Method 3: M+H⁺=544.4, retention time=3.41 min.

### Preparation 11

### Preparation of (2R)-N-benzyl-6-iodochromane-2-carboxamide

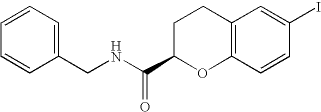

To a solution of (2R)-6-iodo-3,4-dihydro-2H-chromene-2-carboxylic acid (Preparation 1, 5.00 g, 16.4 mmol), benzylamine (1.98 mL, 18.1 mmol) and 1-hydroxybenzotriazole (4.44 g, 32.89 mmol) in DMF (150 mL) was added 1-(3-dimethylaminopropyl)-3-ethylcarbodiimide hydrochloride (6.3 g, 32.9 mmol). After being stirred at room temperature for 2 days, the reaction mixture was concentrated by evaporation. The residue was partitoned between EtOAc and water. The organic layer was separated, washed successively with 1 N HCl and sat. NaHCO₃, dried over Na₂SO₄ and concentrated in vacuo. The residual solid was triturated with isopropyl ether to provide the product as an ivory powder (5.96 g, 92%).

### Preparation 12

### Preparation of N-benzyl-1-[(2R)-6-iodo-3,4-dihydro-2H-chromen-2-yl]methanamine

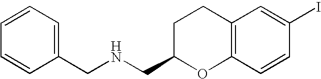

To a solution of (2R)-N-benzyl-6-iodochromane-2-carboxamide (Preparation 11, 7.8 g, 19.9 mmol) in THF (50 mL) was added dropwise BH₃-Me₂S complex (2 M in THF, 100 mL, 200 mmol), and the mixture was refluxed for 2 h. After being cooled, the reaction was quenched through the careful addition of EtOH (25 mL) and 2 N HCl (100 mL). The resultant mixture was stirred at room temperature for 30 min and refluxed for 1 h. The solution was cooled, the volatiles were evaporated off, and the mixture was made basic with aq. NaOH. The aqueous phase was extracted with EtOAc. The organic extract was washed with brine, dried over Na₂SO₄, and concentrated in vacuo to give the crude product as a gum (8.28 g, >99%) that was used without further purification.

### Preparation 13

### Preparation of (2)-1-(benzyl{[(2R)-6-iodo-3,4-dihydro-2H-chromen-2-yl]methyl}amino)-3-phenoxypropan-2-ol

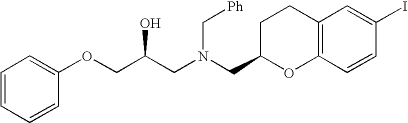

A solution of N-benzyl-1-[(2R)-6-iodo-3,4-dihydro-2H-chromen-2-yl]methanamine (Preparation 12, 8.28 g, 21.8 mmol) and (2S)-2-(phenoxymethyl)oxirane (Preparation 2, 3.28 g, 21.8 mmol) in CH₃CN (100 mL) was stirred at reflux for 5 days. The volatiles were removed and the residue was purified by silica column chromatography (eluant: hexanes/EtOAc, gradient 9:1-4:1) to provide the product (6.4 g, 55%) as a gum.

### Preparation 14

### Preparation of (2S)-N-benzyl-2-{[tert-butyl(dimethyl)silyl]oxy}—N—{[(2R)-6-iodo-3,4-dihydro-2H-chromen-2-yl]methyl}-3-phenoxypropan-1-amine

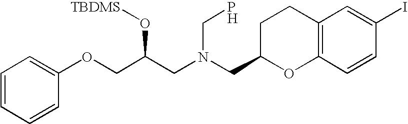

A mixture of (2S)-1-(benzyl {[(2R)-6-iodo-3,4-dihydro-2H-chromen-2-yl]methyl}amino)-3-phenoxypropan-2-ol (Preparation 13, 6.4 g, 12.1 mmol), tert-butyldimethylsilyl chloride (2.36 g, 15.7 mmol), and imidazole (2.06 g, 30.2 mmol) dissolved in CH₂Cl₂ (150 mL) was stirred at room temperature for 2 days. Since a small amount of Preparation 13 remained, additional tert-butyldimethylsilyl chloride (0.80 g, 5.3 mmol) was added, and the stirring was continued for another 2 days. Water was added to the mixture, and the organic layer was separated, washed successively with 1 N HCl, sat. NaHCO₃ and brine, dried over Na₂SO₄, and concentrated in vacuo. The residue was purified by silica column chromatography (eluant: hexanes/EtOAc 19:1) to give the product (7.96 g, >99%) as a colorless syrup.

### Preparation 15

### Preparation of (2S)-N-benzyl-2-{[tert-butyl(dimethyl)silyl]oxy}-3-phenoxy-N-{[(2R)-6-(4,4,5,5-tetramethyl-1,3,2-dioxaborolan-2-yl)-3,4-dihydro-2H-chromen-2-yl]methyl}propan-1-amine

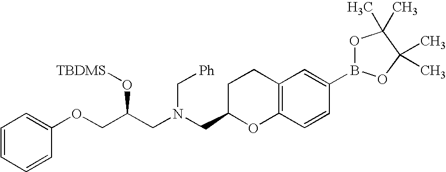

A mixture of (2S)-N-benzyl-2-{[tert-butyl(dimethyl)silyl]oxy}-N-{[(2R)-6-iodo-3,4-dihydro-2H-chromen-2-yl]methyl}-3-phenoxypropan-1-amine (Preparation 14, 100 mg, 0.16 mmol), bis(pinacolate)borane (67.1 mg, 0.264 mmol), potassium acetate (45.7 mg, 0.466 mmol), and 1,1′-bis-(diphenylphosphino)ferrocenepalladium(II) chloride (3.4 mg, 0.005 mmol) in dimethylsulfoxide (7 mL) was stirred at 85° C. overnight. The mixture was partitioned between water and EtOAc. The organic layer was separated, dried and concentrated. The residue was purified by silica column chromatography (eluant: hexanes/EtOAc 9:1) to furnish the product as a gum (40 mg, 40%).

### Preparation 16

### Preparation of (2R)-2-{[benzyl((2S)-2-{[tert-butyl(dimethyl)silyl]oxy}-3-phenoxypropyl)amino]-methyl}chroman-6-ol

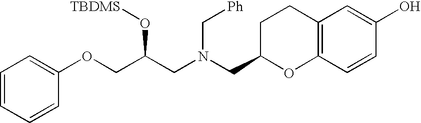

A mixture of (2S)-N-benzyl-2-{[tert-butyl(dimethyl)silyl]oxy}-3-phenoxy-N-{[(2R)-6-(4,4,5,5-tetramethyl-1,3,2-dioxaborolan-2-yl)-3,4-dihydro-2H-chromen-2-yl]methyl}propan-1-amine (Preparation 15, 3.0 g, 4.6 mmol) and N-methylmorpholine-N-oxide (1.64 g, 13.9 mmol) in THF (40 mL) was stirred at 80° C. for 3.5 h. Water was added, and the mixture was extracted with EtOAc. The organic extract was washed with brine, dried over Na₂SO₄ and evaporated. The residue was purified by silica column chromatography (eluant: hexanes/EtOAc gradient 9:1-4:1) to provide the product as a yellow oil (2.7 g, >99%).

### Preparation 17

### Preparation of (2R)-6-iodochromane-2-carboxamide

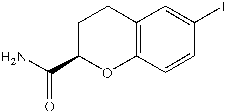

(2R)-6-Iodochromane-2-carboxylic acid (Preparation 1, 10.0 g, 32.9 mmol) and N,N′-carbonyldiimidazole (6.4 g, 39.4 mmol) in DMF (150 mL) were stirred at room temperature for 1.5 h. To this solution was added ammonium acetate (7.6 g, 98.7 mmol), and the mixture was stirred for an additional 2.5 h to complete the reaction. The reaction mixture was cooled to 0° C. and quenched with 160 mL of water. The resultant suspension was then stirred overnight. The white powder was collected by vacuum fitration, washed with water, and dried to give desired product as a white powder in 94% yield (9.4 g).

### Preparation 18

### Preparation of 1-[(2R)-6-iodo-3,4-dihydro-2H-chromen-2-yl]methanamine hydrochloride

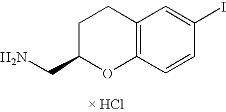

To a suspension of (2R)-6-iodochromane-2-carboxamide (Preparation 17, 9.4 g, 31.0 mmol) in anhydrous THF (75 mL) at reflux was added BH₃-Me₂S complex (2 M in THF, 30 mL, 60 mmol) dropwise. This solution was stirred for 1.5 h, and additional BH₃-Me₂S complex (2 M in THF, 28 mL, 56 mmol) was added. After stirring for 1.5 h, the mixture was cooled to 0° C. and quenched with dropwise addition of MeOH. The mixture was concentrated to 40% of volume and treated with HCl (1 N in Et₂O, 100 mL), producing a white precipitate, which was collected by filtration, washed with ether, and dried to give desired compound as white powder in 76% yield (7.7 g).

### Preparation 19

### Preparation of tert-butyl{[(2R)-6-iodo-3,4-dihydro-2H-chromen-2-yl]methyl}carbamate

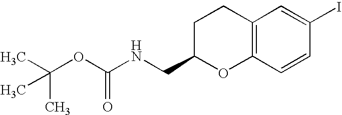

To a suspension of 1-[(2R)-6-iodo-3,4-dihydro-2H-chromen-2-yl]methanamine hydrochloride (Preparation 18, 7.1 g, 21.7 mmol) in THF (35 mL) was added NaHCO₃ (1.8 g, 21.7 mmol) in water (3.5 mL), and the mixture was stirred for 10 min. To this solution was added (tert-BuOCO)₂O (4.7 g, 21.7 mmol), and the mixture was stirred for 2 h. After removing the solvent, the residue was partitioned between water and EtOAc. The organic layer was separated, washed with brine, dried with anhydrous Na₂SO₄, and concentrated to give the desired product as a white solid in quantitative yield (8.5 g).

### Preparation 20

### Preparation of ethyl 2-[((2R)-2-{[(tert-butoxycarbonyl)amino]methyl}-3,4-dihydro-2H-chromen-6-yl)oxy]benzoate

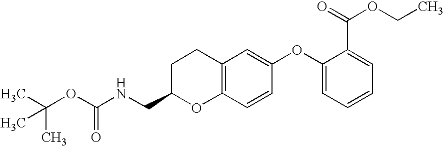

A mixture of tert-butyl {[(2R)-6-iodo-3,4-dihydro-2H-chromen-2-yl]methyl}carbamate (Preparation 19, 6.6 g, 17.0 mmol), ethyl salicylate (6.2 g, 37.4 mmol), 2,2,6,6-tetramethylheptane-3,5-dione (0.9 g, 5.1 mmol), and cesium carbonate (12.7 g, 39.1 mmol) in anhydrous NMP (35 mL) was degassed and filled with argon once. After adding CuCl (1.6 g, 17.0 mmol), the mixture was degassed and filled with argon three times. The mixture was stirred at 120° C. under argon for 3 h. The reaction was diluted with EtOAc (200 mL) and filtered. The filtrate was washed with 2 M HCl, 0.6 M HCl, 2 M NaOH, and 10% NaCl, dried over MgSO₄ and concentrated in vacuo. The residue was further purified through column chromatography (eluant: EtOAc/hexane gradient, 1:3-1:1) to give the desired product in 28% yield (2.0 g).

### Preparation 21

### Preparation of ethyl 2-{[(2R)-2-(aminomethyl)-3,4-dihydro-2H-chromen-6-yl]oxy}benzoate hydrochloride

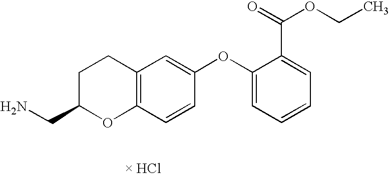

To a solution of ethyl 2-[((2R)-2-{[(tert-butoxycarbonyl)amino]methyl}-3,4-dihydro-2H-chromen-6-yl)oxy]benzoate (Preparation 20, 1.7 g, 4.0 mmol) in CH₂Cl₂ (8 mL) was added HCl (4 N in dioxane, 4 mL, 16 mmol), and the mixture was stirred at room temperature for 2 h. Diethyl ether (15 mL) was added to the reaction mixture, and the resultant precipitate was collected by filtration and dried to give the product in 76% yield (1.1 g).

### Preparation 22

### Preparation of 2-bromo-5-fluorobenzoic Acid

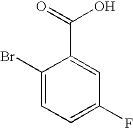

A suspension of 2-amino-5-fluorobenzoic acid (0.465 g, 3.0 mmol) in 48% aq. HBr (2.25 mL) was added to NaNO₂ (0.21 g, 3.15 mmol) dissolved in 0.65 mL of water at 0° C. The resulting solution was treated with CuBr (0.28 g, 1.98 mmol) dissolved in 0.5 mL of 48% aq. HBr, and the mixture was heated at 100° C. for 1 h. After cooling to room temperature, the mixture was extracted with ether three times. The combined organic extracts were washed with brine, dried over MgSO₄, and concentrated in vacuo to give the crude material as white solid. Recrystalization from cyclohexane/EtOAc 15:1 gave the desired product as white crystals in 73% yield (0.483 g). LC-MS, Method 4: M+H⁺=219.0, retention time=1.59 min.

### PREPARATION OF EXAMPLES

### Example I

### Preparation of methyl 2-[((2R)-2-{[(tert-butoxycarbonyl)((2S)-2-{[tert-butyl(dimethyl)silyl]oxy}-3-phenoxypropyl)amino]methyl}-3,4-dihydro-2H-chromen-6-yl)oxy]benzoate

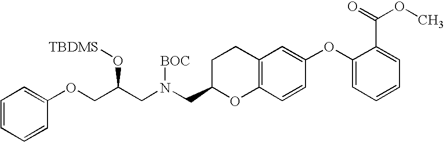

A mixture of methyl salicylate (102 mg, 0.61 mmol) and cesium carbonate (199.4 mg, 0.61 mmol) in NMP (2 mL) was degassed and filled with argon three times. tert-Butyl(2S)-2-{[tert-butyl-(dimethyl)silyl]oxy}-3-phenoxypropyl{[(2R)-6-iodo-3,4-dihydro-2H-chromen-2-yl]methyl}carbamate (Preparation 8, 200 mg, 0.31 mmol) and 2,2,6,6-tetramethylheptane-3,5-dione (11.3 mg, 0.06 mmol) were added followed by copper(I) chloride (15.1 mg, 0.15 mmol). The resulting mixture was degassed and filled with argon three times and then warmed to 120° C. The reaction was stirred at 120° C. overnight. After cooling to room temperature, the slurry was filtered through a Celite pad and washed with EtOAc. Water was added to the filtrate and the product was extracted with EtOAc (two times). The combined organic extract was dried over MgSO₄, concentrated in vacuo, and the residue was purified by preparative TLC (eluant: hexanes/EtOAc 6:1) to give the desired product as clear viscous oil in 19.2% yield (40 mg).

### Preparation of methyl 2-{[(2R)-2-({[(2S)-2-hydroxy-3-phenoxypropyl]amino}methyl)-3,4-dihydro-2H-chromen-6-yl]oxy}benzoate

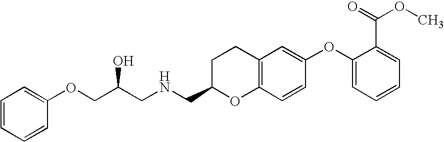

To methyl 2-[((2R)-2-{[(tert-butoxycarbonyl)((2S)-2-{[tert-butyl(dimethyl)silyl]oxy}-3-phenoxypropyl)amino]methyl}-3,4-dihydro-2H-chromen-6-yl)oxy]benzoate (38 mg, 0.056 mmol) was added 4 N HCl in dioxane (3 mL). The reaction mixture was stirred at room temperature for 3 hours and then concentrated under reduced pressure. The residue was purified by preparative TLC (eluant: CHCl₃/MeOH 95:5) to give the desired product as clear oil in 95% yield (24.7 mg).

### Preparation of 2-{[(2R)-2-({[(2S)-2-hydroxy-3-phenoxypropyl]amino}methyl)-3,4-dihydro-2H-chromen-6-yl]oxy}benzoic Acid

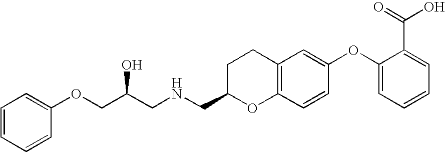

To methyl 2-{[(2R)-2-({[(2S)-2-hydroxy-3-phenoxypropyl]amino}methyl)-3,4-dihydro-2H-chromen-6-yl]oxy}benzoate (22 mg, 0.048 mmol) in THF (4 mL), MeOH (1 mL) and water (1 mL) was added LiOH monohydrate (20 mg, 0.475 mmol). The resulting reaction mixture was stirred at room temperature overnight. The mixture was concentrated in vacuo and 1 N HCl solution was added until precipitation occurred. The precipitate was collected and dried in vacuo to give the desired compound (Example 1) in 68.8% yield (14.7 mg).

Melting point: 188° C.

Molecular weight: 449.508; MS (M+H)⁺450

¹H-NMR (d₆-DMSO): δ 1.63-1.76 (m, 1H), 2.01-2.06 (m, 1H), 2.69-2.87 (m, 21), 3.08 (dd, J=12.4, 9.6, 1), 3.11-3.21 (m, 1H), 3.94-4.03 (m, 2H), 4.28 (bd, J=5.7, 1H), 4.41 (bt, J=9.4, 1H), 5.89 (bs, 1H), 6.75-6.77 (m, 2H), 6.81-6.83 (m, 1H), 6.88 (d, J=5.5, 1H), 6.94-6.98 (m, 3H), 7.18 (dt, J=7.4, 1.1, 1H), 7.31 (t, J=8.2, 2H), 7.50 (dt, J=8.3, 1.7, 1H), 7.77 (dd, J=7.7, 1.7, 1H), 8.95 (bs, 1H).

### Example 2

### Preparation of methyl 4-[((2R)-2-{[benzyl((2S)-2-{[tert-butyl(dimethyl)silyl]oxy}-3-phenoxypropyl)amino]methyl}-3,4-dihydro-2H-chromen-6-yl)amino]benzoate

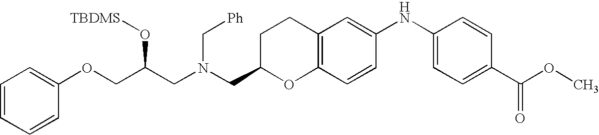

Argon was bubbled through a mixture of methyl 4-aminobenzoate (70.45 mg, 0.47 mmol), dicyclohexyl(2′,4′,6′-triisopropylbiphenyl-2-yl)phosphine (22.22 mg, 0.05 mmol), Pd(OAc)₂ (3.49 mg, 0.02 mmol) in toluene (3 mL) in a sealed tube for 10 minutes before addition of (2S)-N-benzyl-2-{[tert-butyl(dimethyl)silyl]oxy}-N-{[(2R)-6-iodo-3,4-dihydro-2H-chromen-2-yl]methyl}-3-phenoxypropan-1-amine (Preparation 14, 200 mg, 0.31 mmol) in toluene (1 mL) and tert-BuOH (1 mL). The reaction mixture was degassed with argon flow, the tube was capped and heated at 100° C. for 2 hours. After cooling to room temperature, the mixture was filtered though a Celite pad and washed with EtOAc. The filtrate was concentrated in vacuo, and the residue was purified by column chromatography (eluant: gradient 100% hexanes-hexanes/EtOAc 9:1) to give the product in 89.5% yield (185.40 mg).

### Preparation of methyl 4-[((2R)-2-{[benzyl((2S)-2-{[tert-butyl(dimethyl)silyl]oxy}-3-phenoxypropyl)amino]methyl}-3,4-dihydro-2H-chromen-6-yl)(methyl)amino]benzoate

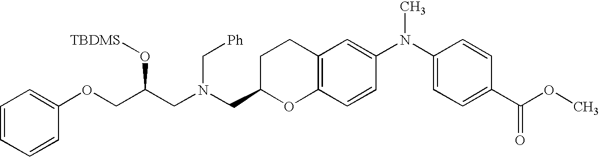

To a mixture of methyl 4-[((2R)-2-{[benzyl((2S)-2-{[tert-butyl(dimethyl)silyl]oxy}-3-phenoxypropyl)amino]methyl}-3,4-dihydro-2H-chromen-6-yl)amino]benzoate (185 mg, 0.28 mmol) in THF (3 mL) were added MeI (0.155 mL, 2.50 mmol) followed by NaH (60% purity, 49.93 mg, 1.25 mmol) at 0° C. The mixture was stirred at room temperature for 3 hours. The reaction was quenched by addition of sat. NH₄Cl solution and then extracted with EtOAc. The organic layer was dried over MgSO₄, concentrated in vacuo, and the residue was purified by column chromatography (eluant: gradient 100% hexanes—hexanes/EtOAc 9:1) to give the product in 87.6% yield (165.4 mg).

### Preparation of methyl 4-[((2R)-2-{[((2S)-2-{[tert-butyl(dimethyl)silyl]oxy}-3-phenoxypropyl)-amino]methyl}-3,4-dihydro-2H-chromen-6-yl)(methyl)amino]benzoate

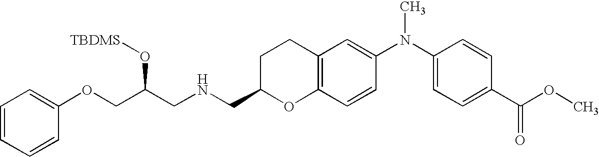

To a solution of methyl 4-[((2R)-2-{[benzyl((2S)-2-{[tert-butyl(dimethyl)silyl]oxy}-3-phenoxypropyl)amino]methyl}-3,4-dihydro-2H-chromen-6-yl)(methyl)amino]benzoate (160 mg, 0.23 mmol) in MeOH/CH₂Cl₂ (3 mL) was added Pd(OH)₂ (50 mg), and the mixture was charged with H₂ gas using a balloon. After stirring for 5 hours, the catalyst was removed by filtration and the filtrate was concentrated in vacuo. The crude product (144.30 mg, >99%) was used in the next reaction without further purification.

### Preparation of methyl 4-[[(2R)-2-({[(2S)-2-hydroxy-3-phenoxypropyl]amino}methyl)-3,4-dihydro-2H-chromen-6-yl](methyl)amino]benzoate Hydrochloride

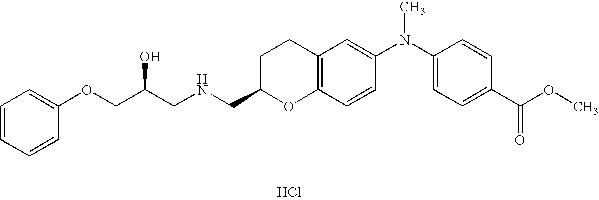

The crude methyl 4-[((2R)-2-{[((2S)-2-{[tert-butyl(dimethyl)silyl]oxy}-3-phenoxypropyl)amino]-methyl}-3,4-dihydro-2H-chromen-6-yl)(methyl)amino]benzoate (140 mg, 0.24 mmol) was dissolved in 4 N HCl in dioxane (3 mL), and the mixture was stirred at room temperature for 2 hours. After evaporation of the solvent, the resulting precipitate was collected by filtration and washed with hexane. The product (78% yield, 95.70 mg) was dried in vacuo.

### Preparation of 4-[[(2R)-2-({[(2S)-2-hydroxy-3-phenoxypropyl]amino}methyl)-3,4-dihydro-2H-chromen-6-yl](methyl)amino]benzoic acid hydrochloride

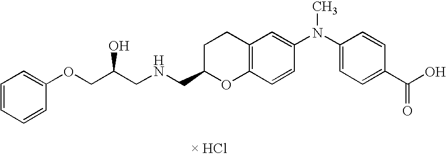

To a solution of methyl 4-[[(2R)-2-({[(2S)-2-hydroxy-3-phenoxypropyl]amino}methyl)-3,4-dihydro-2H-chromen-6-yl](methyl)amino]benzoate (84.0 mg, 0.16 mmol) in MeOH (1 mL) was added 1 N NaOH (0.50 mL). The resulting mixture was refluxed at 80° C. for 3 hours with stirring. After cooling to room temperature, 4 N HCl in dioxane was added dropwise until precipitation occurred. The resulting precipitate was collected by filtration and washed with Et₂O. The residue was collected and dried in vacuo to give the resired product in 84% yield (69.0 mg).

Melting point: 288° C. (dec.)

Molecular weight: 499.012; MS (M+H)⁺: 500.

The following compounds were prepared in a similar manner as described in Example 1 or Example 2:

TABLE 1

| | | | | MS | |
|---|---|---|---|---|---|
| | | | | (M + H)⁺ | |
| Example | | Preparation | | (LC-MS | M.p. |
| No. | Structure | Method | MW | Method) | [° C.] |
| 3 | 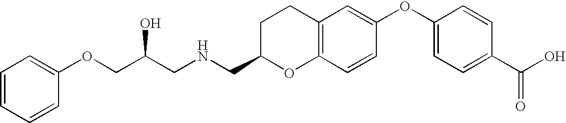 | Example 1 | 450 | 450 (1) | 154.2 |
| 4 | 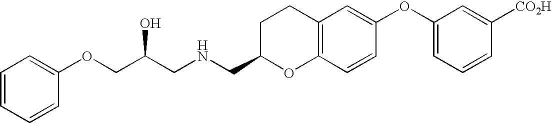 | Example 1 | 450 | 450 (1) | 214 |
| 5 | 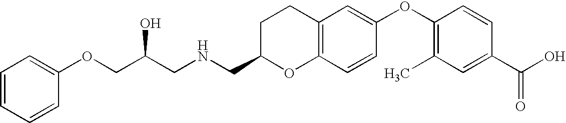 | Example 1 | 464 | 464 (1) | 131.3 |
| 6 | 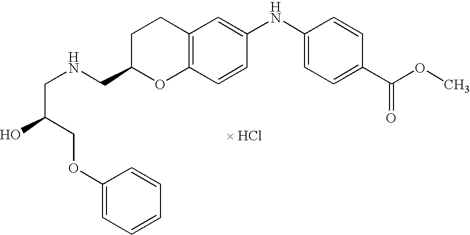 | Example 2 | 499 | 500 (1) | 238 |
| 7 | 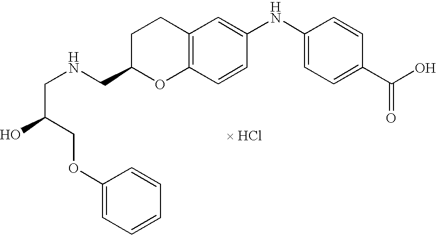 | Example 2 | 485 | 485 (1) | 268 |
| 8 | 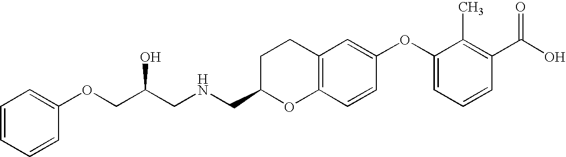 | Example 1 | 464 | 464 (1) | 265 |
| 9 | 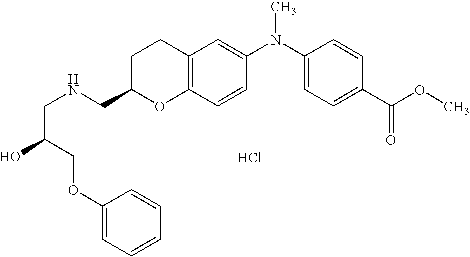 | Example 2 | 513 | 514 (1) | 229 (dec.) |
| 10 | 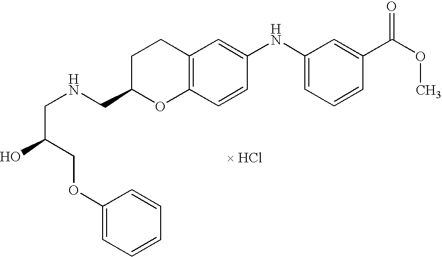 | Example 2 | 499 | 500 (1) | 170 |
| 11 | 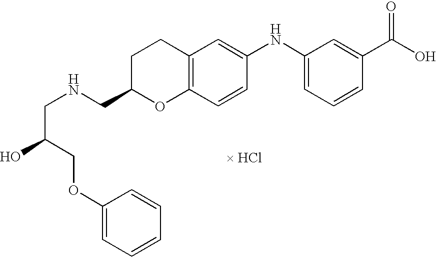 | Example 2 | 485 | 485 (1) | 274 (dec.) |
| 12 | 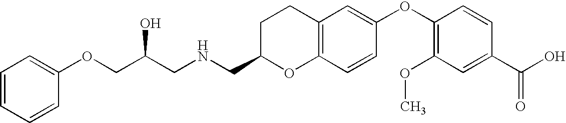 | Example 1 | 480 | 480 (1) | 238.1 |
| 13 | 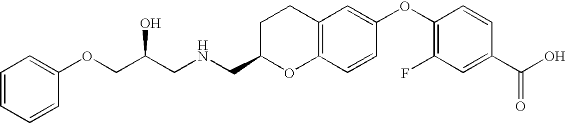 | Example 1 | 467 | 468 (1) | 251.1 |
| 14 | 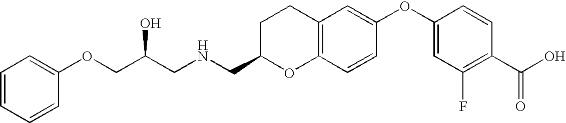 | Example 1 | 467 | 468 (1) | 258.7 |
| 15 | 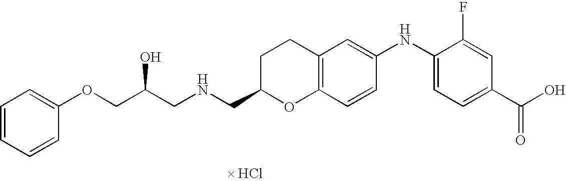 | Example 2 | 503 | 503 (1) | 197 |
| 16 | 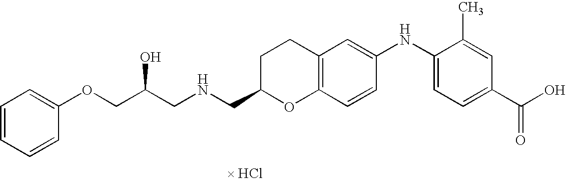 | Example 2 | 499 | 500 (1) | 208 |
| 17 | 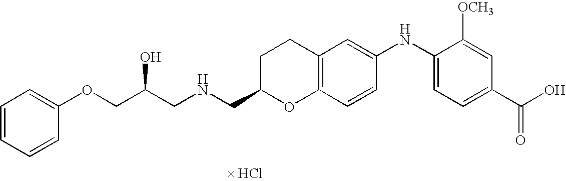 | Example 2 | 515 | 515 (1) | 210 |
| 18 | 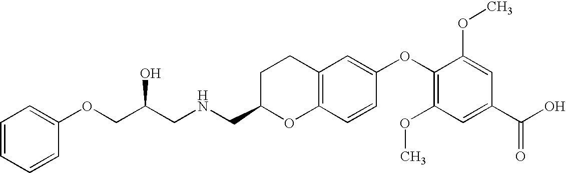 | Example 1 | 510 | 510 (1) | 231.3 |
| 19 | 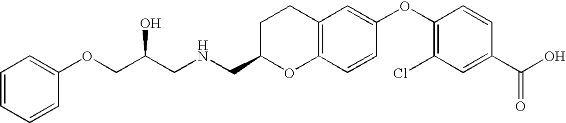 | Example 1 | 484 | 484 (1) | 232.7 |
| 20 | 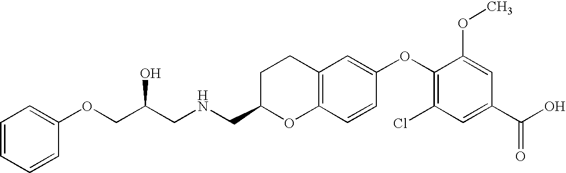 | Example 1 | 514 | 514 (1) | 239.1 |
| 21 | 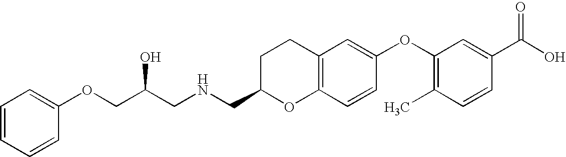 | Example 1 | 464 | 464 (1) | 266 |
| 22 | 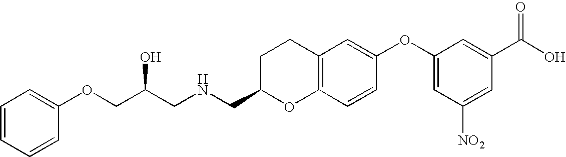 | Example 1 | 495 | 495 (1) | 213 |
| 23 | 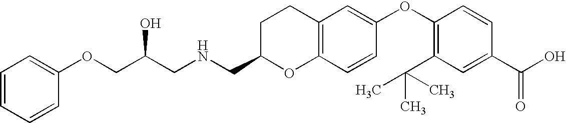 | Example 1 | 506 | 506 (1) | 216.2 |
| 24 | 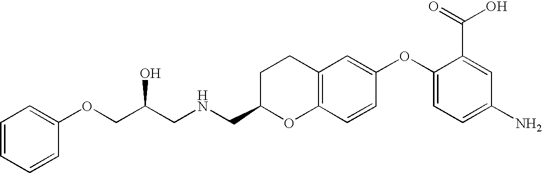 | Example 1 | 537 | 538 (1) | 137 |
| 25 | 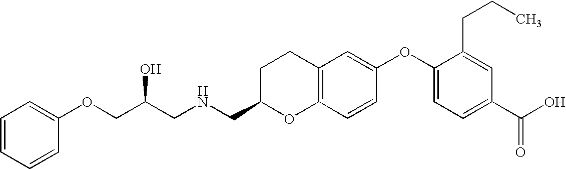 | Example 1 | 492 | 492 (1) | 221 |
| 26 | 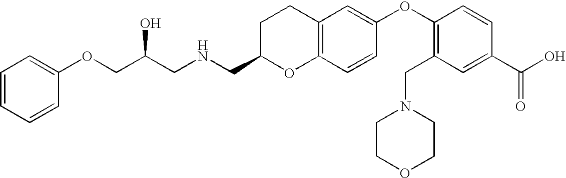 | Example 1 | 549 | | |
| 27 | 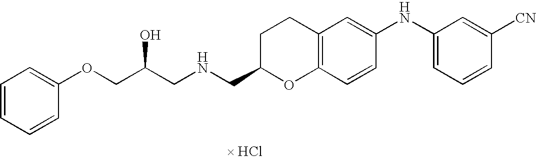 | Example 2 | 466 | | 182 |
| 28 | 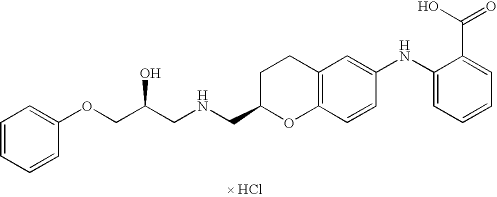 | Example 2 | 485 | | 203 |
| 29 | 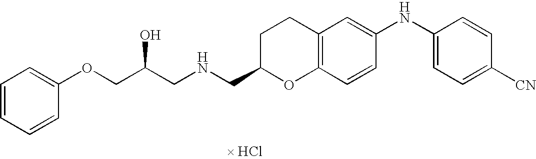 | Example 2 | 466 | | 196 |
| 30 | 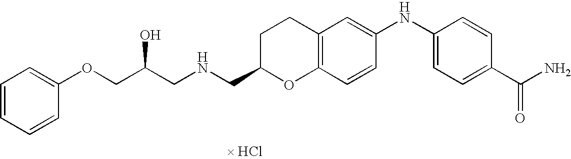 | Example 2 | 484 | | 228 |
| 31 | 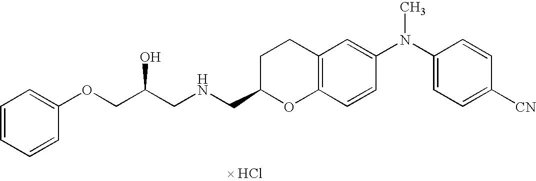 | Example 2 | 480 | | 215 |
| 32 | 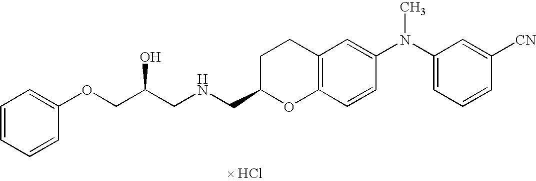 | Example 2 | 480 | | 171 |
| 33 | 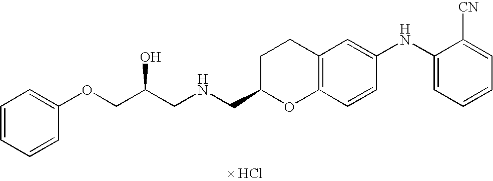 | Example 2 | 466 | | 203 |
| 34 | 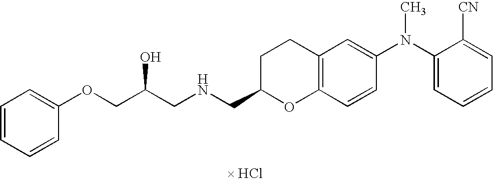 | Example 2 | 480 | | 187 |

### Example 35

### Preparation of ethyl 2-{[(2R)-2-({[(2S)-3-(9H-carbazol-4-yloxy)-2-hydroxypropyl]amino}methyl)-3,4-dihydro-2H-chromen-6-yl]oxy}benzoate

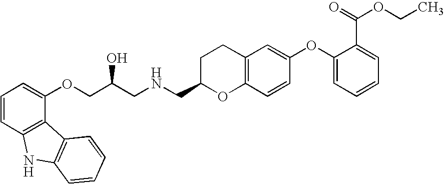

To a solution of ethyl 2-{[(2R)-2-(aminomethyl)-3,4-dihydro-2H-chromen-6-yl]oxy}benzoate hydrochloride (Preparation 21, 30.0 mg, 0.082 mmol) in 0.3 mL of dioxane was added Et₃N (0.011 mL, 0.082 mmol), followed by 4-[(2S)-oxiran-2-ylmethoxy]-9H-carbazole (prepared in analogy to Preparation 2; 19.7 mg, 0.082 mmol) and K₂CO₃ (22.8 mg, 16.5 mmol). The mixture was stirred overnight at 135° C. After removing the solvent, the residue was purified through preparative TLC (eluant: CH₂Cl₂/MeOH 10:1) to give the desired product in 30% yield (14.0 mg).

### Preparation of 2-{[(2R)-2-({[(2S)-3-(9H-carbazol-4-yloxy)-2-hydroxypropyl]amino}methyl)-3,4-dihydro-2H-chromen-6-yl]oxy}benzoic Acid Hydrochloride

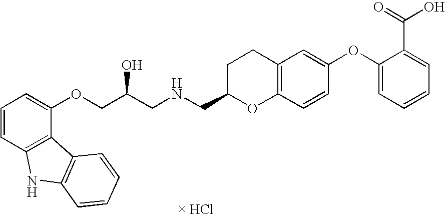

To a solution of ethyl 2-{[(2R)-2-({[(2S)-3-(9H-carbazol-4-yloxy)-2-hydroxypropyl]amino}-methyl)-3,4-dihydro-2H-chromen-6-yl]oxy}benzoate (14.0 mg, 0.025 mmol) in THF (0.5 mL) was added LiOH (1 N aqueous solution, 0.5 mL, 0.5 mmol), and the mixture was stirred at 50° C. overnight. After removing the solvent, the residue was triturated with 1 N HCl, producing a white precipitate. The precipitate was collected, washed with water, and dried in an oven to give the desired product as brownish powder in 85% yield (12.1 mg).

Melting point: 186° C.

The following compounds were prepared in a similar manner as described in Example 35:

TABLE 2

| Example | | | M.p. |
|---|---|---|---|
| No. | Structure | MW | [° C.] |
| 36 | 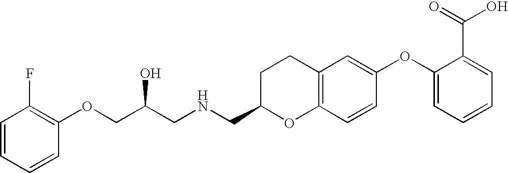 | 467 | 209 |
| 37 | 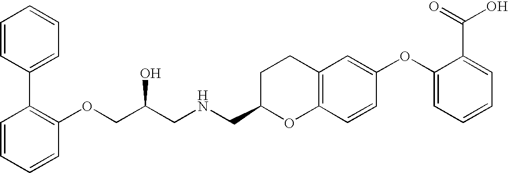 | 526 | 175 |
| 38 | 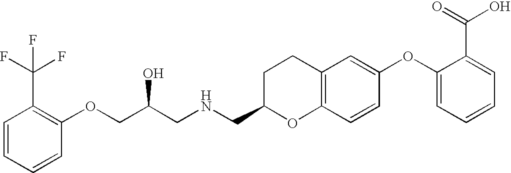 | 517 | 162 |
| 39 | 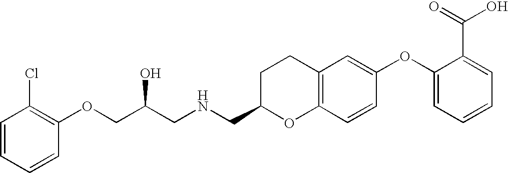 | 484 | 230 |
| 40 | 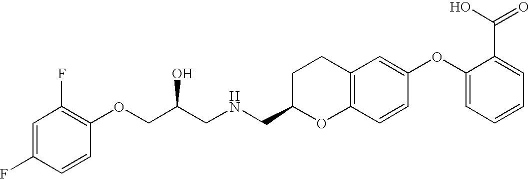 | 485 | 138-142 |
| 41 | 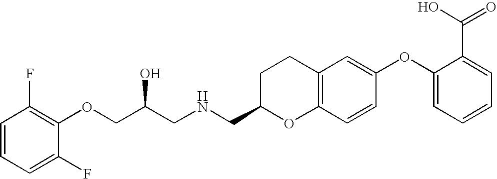 | 485 | 196-198 |
| 42 | 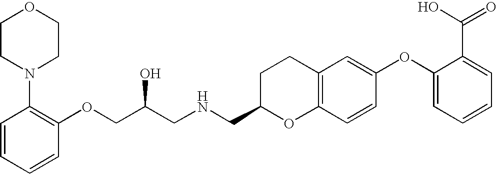 | 535 | 173-176 |
| 43 | 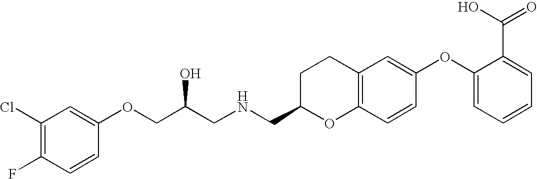 | 502 | 196-199 |

### Example 44

### Preparation of 2-[((2R)-2-{[(tert-butoxycarbonyl)((2S)-2-{[tert-butyl(dimethyl)silyl]oxy}-3-phenoxypropyl)amino]methyl}-3,4-dihydro-2H-chromen-6-yl)oxy]-5-fluorobenzoic Acid

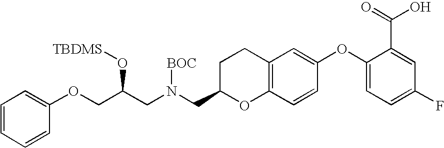

Argon was bubbled through a mixture of tert-butyl((2S)-2-{[tert-butyl(dimethyl)silyl]oxy}-3-phenoxypropyl) {[(2R)-6-hydroxy-3,4-dihydro-2H-chromen-2-yl]methyl}carbamate (Preparation 10, 100 mg, 0.184 mmol), 2-bromo-5-fluorobenzoic acid (Preparation 22, 40.3 mg, 0.184 mmol), N,N-dimethyl-4-aminopyridine (44.9 mg, 0.368 mmol), copper(II) oxide (21.9 mg, 0.276 mmol), and copper powder (17.5 mg, 0.276 mmol) in acetonitrile (3.5 mL) at room temperature, and then the reaction mixture was heated at reflux for 3 hours. At this point, additional N,N-dimethyl-4-amino-pyridine (44.9 mg, 0.368 mmol), copper(II) oxide (21.9 mg, 0.276 mmol), and copper powder (17.5 mg, 0.276 mmol) were added to the mixture. The mixture was maintained at reflux overnight. After cooling to room temperature, the mixture was diluted with CH₂Cl₂ and washed three times with 1 N HCl, washed with brine, dried over MgSO₄, and concentrated in vacuo. The residue was further purified through preparative TLC (eluant: CH₂Cl₂/MeOH 50:1) to give the desired product in 34% yield (43 mg). LC-MS, Method 3: M+H⁺=682.5, retention time=3.54 min.

### Preparation of 5-fluoro-2-{[(2R)-2-({[(2S)-2-hydroxy-3-phenoxypropyl]amino}methyl)-3,4-dihydro-2H-chromen-6-yl]oxy}benzoic Acid Hydrochloride

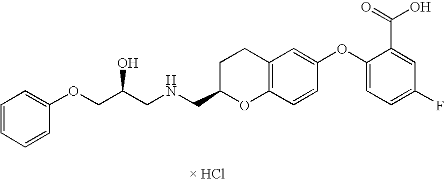

To a solution of 2-[((2R)-2-{[(tert-butoxycarbonyl)((2S)-2-{[tert-butyl(dimethyl)silyl]oxy}-3-phenoxypropyl)amino]methyl}-3,4-dihydro-2H-chromen-6-yl)oxy]-5-fluorobenzoic acid (39.4 mg, 0.058 mmol) in dioxane (0.5 mL) was added HCl (4 N in dioxane, 1.0 mL, 4 mmol), and the mixture was stirred at room temperature overnight. After removing all volatile material, the residue was triturated with ether. The resulting precipitate was collected and dried to give the desired product in 82% yield (23.9 mg). LC-MS, Method 3: M+H⁺=468.3, retention time=1.81 min.

The following compounds were prepared in a similar manner as described in Example 44:

TABLE 3

| Example | | | MS (M + H)⁺ | Retention |
|---|---|---|---|---|
| No. | Structure | MW | (LC-MS Method) | Time [min] |
| 45 | 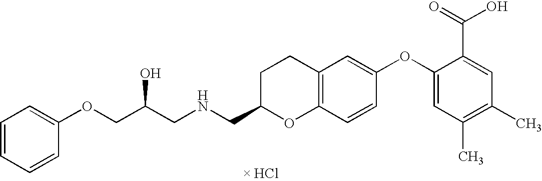 | 514 | 478.3 (3) | 1.91 |
| 46 | 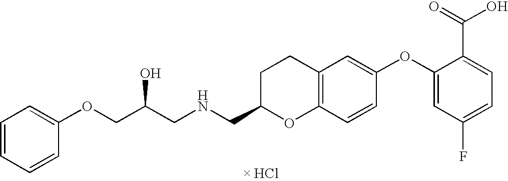 | 504 | 468.3 (3) | 1.78 |
| 47 | 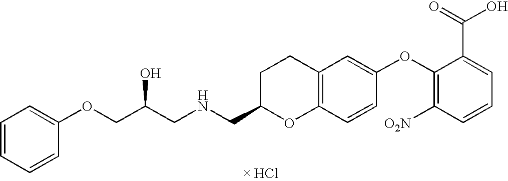 | 531 | 495.4 (3) | 1.75 |
| 48 | 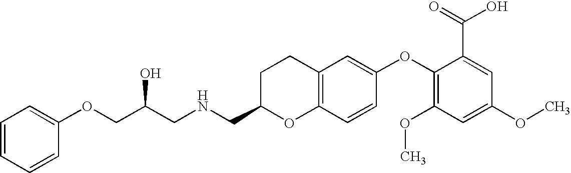 | 510 | 510.1 (2) | 1.68 |
| 49 | 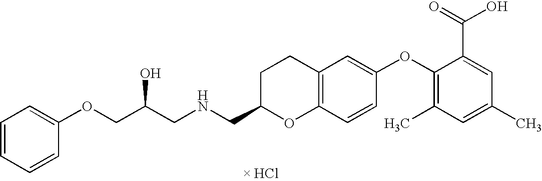 | 514 | 478.4 (3) | 1.92 |
| 50 | 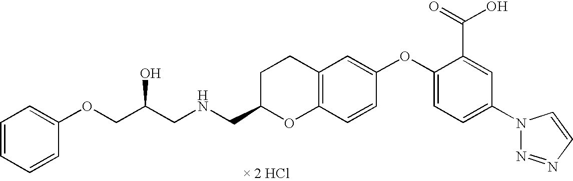 | 589 | 517.4 (3) | 1.66 |
| 51 | 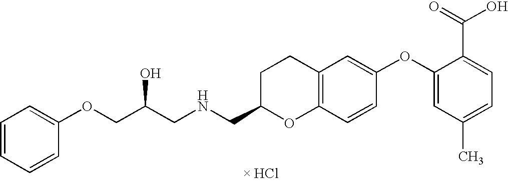 | 500 | 464.4 (3) | 1.84 |
| 52 | 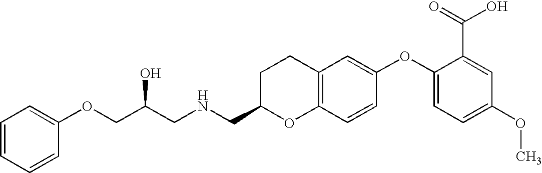 | 480 | 480.4 (4) | 1.74 |
| 53 | 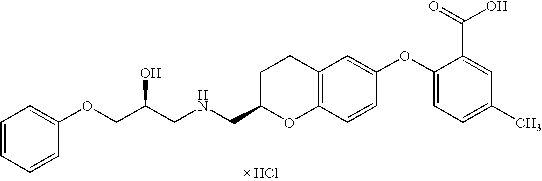 | 500 | 464.4 (4) | 1.88 |
| 54 | 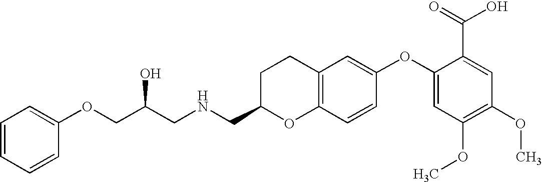 | 520 | 510.4 (3) | 1.7 |
| 55 | 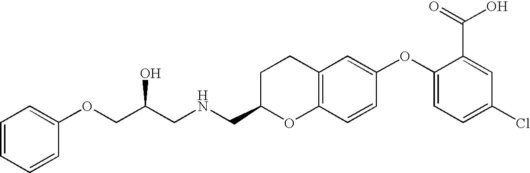 | 520 | 484.4 (3) | 1.91 |
| 56 | 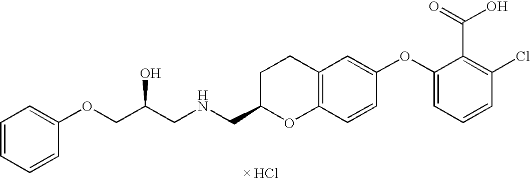 | 520 | 484.3 (3) | 1.81 |
| 57 | 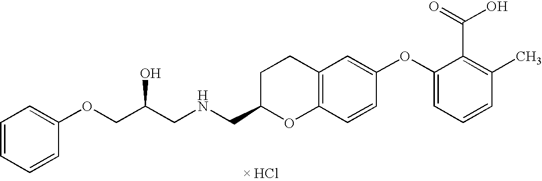 | 500 | 464.4 (2) | 1.64 |
| 58 | 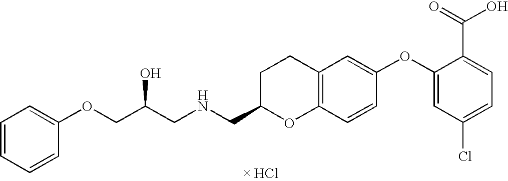 | 520 | 484.3 (4) | 1.94 |
| 59 | 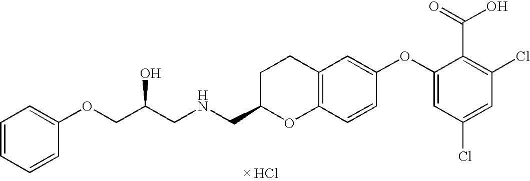 | 555 | 518.3 (4) | 1.96 |
| 60 | 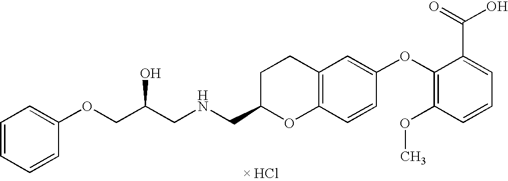 | 516 | 480.3 (4) | 1.79 |
| 61 | 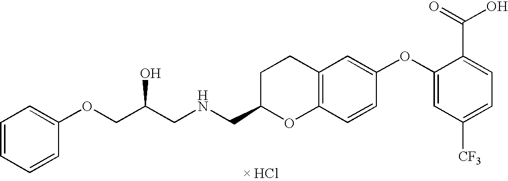 | 554 | 518.4 (4) | 2.01 |
| 62 | 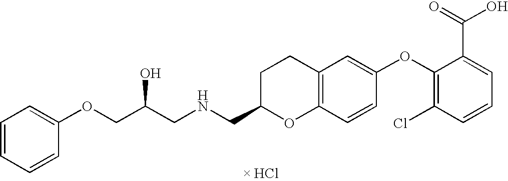 | 520 | 484.2 (4) | 1.96 |
| 63 | 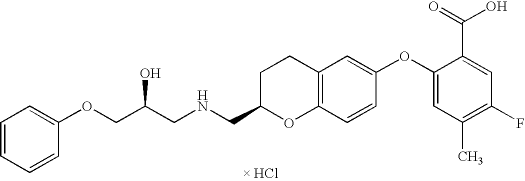 | 518 | 482.3 (4) | 1.93 |
| 64 | 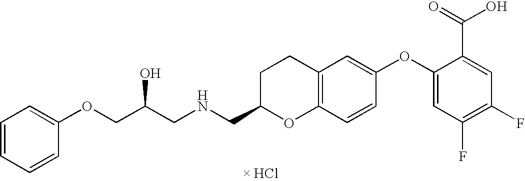 | 522 | 486.2 (4) | 1.92 |
| 65 | 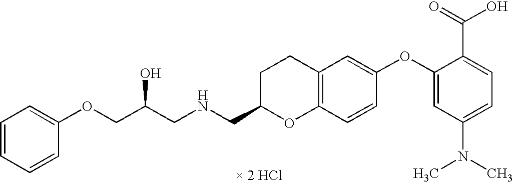 | 565 | 493.3 (4) | 1.86 |
| 66 | 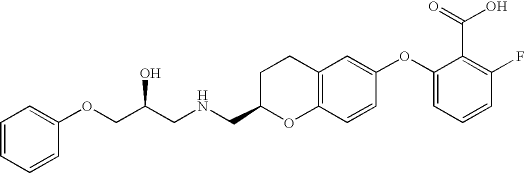 | 467 | 468.3 (4) | 1.79 |
| 67 | 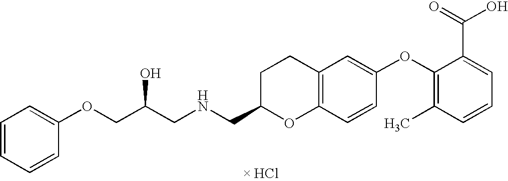 | 500 | 464.1 (2) | 1.77 |
| 68 | 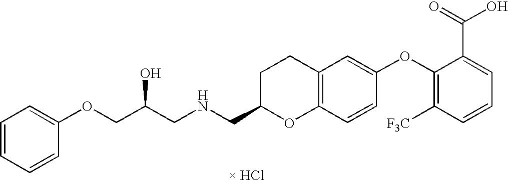 | 554 | 518.0 (2) | 1.87 |
| 69 | 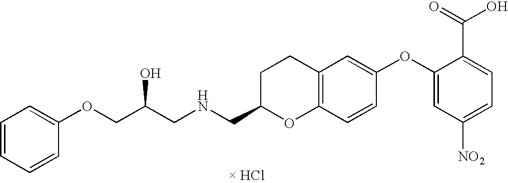 | 495 | 495.3 (4) | 1.86 |

1. An chroman derivative of the formula (I), its tautomeric or stereoisomeric form, or a salt thereof: 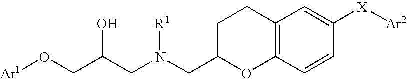
wherein
R¹ represents hydrogen or C₁₋₆ alkyl;
X represents O or NR² (wherein R² represents hydrogen or C₁₋₆ alkyl);
Ar¹ represents phenyl or 5-14 membered heteroaryl containing one, two or three heteroatoms each independently selected from O, S, or N atom
wherein said phenyl or 5-14 membered heteroaryl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁alkoxycarbonyl), benzyl (in which phenyl moiety is optionally substituted by halogen, nitro, hydroxy, carboxy, amino, C₁₋₆ alkylamino, di(C₁₋₆ alkyl) amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, 5-6 membered heteroaryl and heterocyclyl; and
Ar² represents phenyl or 5-6 membered heteroaryl containing one or two heteroatoms each independently selected from O, S, or N atom
wherein said phenyl or 5-6 membered heteroaryl is substituted by one selected from the group consisting of carboxyl, C₁₋₆ alkoxycarbonyl, hydroxycarbonyl C₁₋₆alkyl, hydroxycarbonylC₁₋₆alkyloxy, carbamoyl, cyano and 5-6 membered unsaturated heterocyclyl,
and further substituted by one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆alkylcarbamoyl, cyano, C₁₋₆alkyl (which alkyl is optionally substituted by cyano, nitro, hydroxy, carboxy, amino, C, alkoxycarbonyl, heterocyclyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, 5-6 membered heteroaryl and heterocyclyl.

2. The chroman derivative of the formula (I), its tautomeric or stereoisomeric form, or a salt thereof as claimed in claim 1,
wherein
R¹ represents hydrogen;
X represents O;
Ar¹ represents phenyl
wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)-amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl or 5-6 membered heteroaryl containing one or two hetero-atoms each independently selected from O, S, or N atom
wherein said phenyl or 5-6 membered heteroaryl is substituted by one selected from the group consisting of carboxyl, C₁₋₆ alkoxycarbonyl, hydroxycarbonylC₁₋₆alkyl, hydroxycarbonylC₁₋₆alkyloxy, carbamoyl, tetrazole, 1,2,4-triazole, 5-oxo-1,2,4-oxadiazol, 5-oxo-1,2,4-thiadiazol, 5-thiooxo-1,2,4-oxadiazole, and 1,2,3,5-oxathiadiazole 2-oxide
and further substituted by one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

3. The chroman derivative of the formula (I), its tautomeric or stereoisomeric form, or a salt thereof as claimed in claim 1,
wherein
R¹ represents hydrogen;
X represents NR² (wherein R² represents hydrogen or C₁₋₆ alkyl);
Ar¹ represents phenyl
wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)-amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl or 5-6 membered heteroaryl containing one or two heteroatoms each independently selected from O, S, or N atom
wherein said phenyl or 5-6 membered heteroaryl is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl) amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

4. The chroman derivative of the formula (I), its tautomeric or stereoisomeric form, or a salt thereof as claimed in claim 1,
wherein
R¹ represents hydrogen or C₁₋₆ alkyl;
X represents O or NR² (wherein R² represents hydrogen or C₁₋₆ alkyl);
Ar¹ represents phenyl
wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)-amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl
wherein said phenyl is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

5. The chroman derivative of the formula (I), its tautomeric or stereoisomeric form, or a salt thereof as claimed in claim 1,
wherein
R¹ represents hydrogen;
X represents O or NR² (wherein R² represents hydrogen or C₁₋₆ alkyl);
Ar¹ represents pyridine or pyrimidine
wherein said pyridine or pyrimidine is substituted by one or two substitutents independently selected from the group consisting of hydrogen, halogen, nitro, hydroxy, carboxy, amino, C₁₋₆ alkylamino, di(C₁₋₆ alkyl)-amino, C₃₋₈ cycloalkylamino, C₁₋₆ alkoxycarbonyl, phenyl (which phenyl is optionally substituted by halogen, nitro, hydroxy, carboxy, amino, C₁₋₆ alkyl-amino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents phenyl or 5-6 membered heteroaryl containing one or two heteroatoms each independently selected from O, S, or N atom
wherein said phenyl or 5-6 membered heteroaryl is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxy-carbonyl), benzyl (in which phenyl moiety is optionally substituted by halogen, nitro, hydroxy, carboxy, amino, C₁₋₆ alkylamino, di(C₁₋₆ alkyl) amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

6. The chroman derivative of the formula (I), its tautomeric or stereoisomeric form, or a salt thereof as claimed in claim 1,
wherein
R¹ represents hydrogen or C₁₋₆ alkyl;
X represents O or NR² (wherein R² represents hydrogen or C₁₋₆ alkyl);
Ar¹ represents phenyl
wherein said phenyl is substituted by one or two substitutents independently selected from the group consisting of hydrogen, halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)-amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
Ar² represents pyridine or pyrimidine
wherein said pyridine or pyrimidine is substituted by COOR⁵ (wherein R⁵ represents hydrogen or C₁₋₆ alkyl) and one or two additional substitutents each independently selected from the group consisting of hydrogen, halogen, nitro, hydroxy, carboxy, amino, C₁₋₆ alkylamino, di(C₁₋₆ alkyl)-amino, C₃₋₈ cycloalkylamino, C₁₋₆ alkoxycarbonyl, phenyl (which phenyl is optionally substituted by halogen, nitro, hydroxy, carboxy, amino, C₁₋₆ alkyl-amino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

7. The chroman derivative of the formula (I), its tautomeric or stereoisomeric form, or a salt thereof as claimed in claim 1, wherein said chroman derivative of the formula (I) is selected from the group consisting of:
4-{[(2R)-2-({[(2S)-2-hydroxy-3-phenoxypropyl]amino}methyl)-3,4-dihydro-2H-chromen-6-yl]oxy}benzoic acid;
3-{[(2R)-2-({[(2S)-2-hydroxy-3-phenoxypropyl]amino}methyl)-3,4-dihydro-2H-chromen-6-yl]oxy}benzoic acid;
4-{[(2R)-2-({[(2S)-2-hydroxy-3-phenoxypropyl]amino}methyl)-
3,4-dihydro-2H-chromen-6-yl]oxy}-3-methylbenzoic acid;
methyl 4-{[(2R)-2-({[(2S)-2-hydroxy-3-phenoxypropyl]amino}methyl)-3,4-dihydro-2H-chromen-6-yl]amino}benzoate;
4-{[(2R)-2-({[(2S)-2-hydroxy-3-phenoxypropyl]amino}methyl)-3,4-dihydro-2H-chromen-6-yl]amino}benzoic acid;
3-{[(2R)-2-({[(2S)-2-hydroxy-3-phenoxypropyl]amino}methyl)-3,4-dihydro-2H-chromen-6-yl]oxy}-2-methylbenzoic acid;
methyl 4-[[(2R)-2-({[(2S)-2-hydroxy-3-phenoxypropyl]amino}methyl)-3,4-dihydro-2H-chromen-6-yl](methyl)amino]benzoate;
4-[[(2R)-2-({[(2S)-2-hydroxy-3-phenoxypropyl]amino}methyl)-3,4-dihydro-2H-chromen-6-yl](methyl)amino]benzoic acid;
2-{[(2R)-2-({[(2S)-2-hydroxy-3-phenoxypropyl]amino}methyl)-3,4-dihydro-2H-chromen-6-yl]oxy}benzoic acid;
methyl 3-{[(2R)-2-({[(2S)-2-hydroxy-3-phenoxypropyl]amino}methyl)-3,4-dihydro-2H-chromen-6-yl]amino}benzoate;
3-{[(2R)-2-({[(2S)-2-hydroxy-3-phenoxypropyl]amino}methyl)-3,4-dihydro-2H-chromen-6-yl]amino}benzoic acid;
4-{[(2R)-2-({[(2S)-2-hydroxy-3-phenoxypropyl]amino}methyl)-3,4-dihydro-2H-chromen-6-yl]oxy}-3-methoxybenzoic acid;
b 3-fluoro-4-{[(2R)-2-({[(2S)-2-hydroxy-3-phenoxypropyl]amino}methyl)-3,4-dihydro-2H-chromen-6-yl]oxy}benzoic acid;
2-fluoro-4-{[(2R)-2-({[(2S)-2-hydroxy-3-phenoxypropyl]amino}methyl)-3,4-dihydro-2H-chromen-6-yl]oxy}benzoic acid;
3-fluoro-4-{[(2R)-2-({[(2S)-2-hydroxy-3-phenoxypropyl]amino}methyl)-3,4-dihydro-2H-chromen-6-yl]amino}benzoic acid;
4-{[(2R)-2-({[(2S)-2-hydroxy-3-phenoxypropyl]amino}methyl)-3,4-dihydro-2H-chromen-6-yl]amino}-3-methylbenzoic acid;
4-{[(2R)-2-({[(2S)-2-hydroxy-3-phenoxypropyl]amino}methyl)-3,4-dihydro-2H-chromen-6-yl]amino}-3-methoxybenzoic acid;
4-{[(2R)-2-({[(2S)-2-hydroxy-3-phenoxypropyl]amino}methyl)-3,4-dihydro-2H-chromen-6-yl]oxy}-3,5-dimethoxybenzoic acid;
3-chloro-4-{[(2R)-2-({[(2S)-2-hydroxy-3-phenoxypropyl]amino}methyl)-3,4-dihydro-2H-chromen-6-yl]oxy}benzoic acid;
3-chloro-4-{[(2R)-2-({[(2S)-2-hydroxy-3-phenoxypropyl]amino}methyl)-3,4-dihydro-2H-chromen-6-yl]oxy}-5-methoxybenzoic acid;
3-{[(2R)-2-({[(2S)-2-hydroxy-3-phenoxypropyl]amino}methyl)-3,4-dihydro-2H-chromen-6-yl]oxy}-4-methylbenzoic acid;
3-{[(2R)-2-({[(2S)-2-hydroxy-3-phenoxypropyl]amino}methyl)-3,4-dihydro-2H-chromen-6-yl]oxy}-5-nitrobenzoic acid;
3-tert-butyl-4-{[(2R)-2-({[(2S)-2-hydroxy-3-phenoxypropyl]amino}methyl)-3,4-dihydro-2H-chromen-6-yl]oxy}benzoic acid;
5-amino-2-{[(2R)-2-({[(2S)-2-hydroxy-3-phenoxypropyl]amino}methyl)-3,4-dihydro-2H-chromen-6-yl]oxy}benzoic acid hydrochloride; and
4-{[(2R)-2-({[(2S)-2-hydroxy-3-phenoxypropyl]amino}methyl)-3,4-dihydro-2H-chromen-6-yl]oxy}-3-propylbenzoic acid.

8. A medicament comprising the chroman derivative of the formula (I), its tautomeric or stereoisomeric form, or a physiologically acceptable salt thereof as claimed in claim 1 as an active ingredient.

9. The medicament as claimed in claim 8, further comprising one or more pharmaceutically acceptable excipients.

10. The medicament as claimed in claim 8, wherein said chroman derivative of the formula (I), its tautomeric or stereoisomeric form, or a physiologically acceptable salt thereof is a BETA 3 antagonist.

11. The medicament as claimed in claim 8 for the treatment and/or prevention of an urological disorder or disease.

12. The medicament as claimed in claim 11, wherein said urological disorder or disease is detrusor overactivity (overactive bladder), urinary incontinence, neurogenic detrusor oeractivity (detrusor hyperflexia), idiopathic detrusor overactivity (detrusor instability), benign prostatic hyperplasia, and lower urinary tract symptoms.

13. The medicament as claimed in claim 8 for the treatment and/or prevention of an inflammatory disorder or disease.

14. The medicament as claimed in claim 13, wherein said inflammatory disorder or disease is asthma or COPD.

15. Use of compounds according to claim 1 for manufacturing a medicament for the treatment and/or prevention of an urological disorder or disease.

16. Use of compounds according to claim 1 for manufacturing a medicament for the treatment and/or prevention of an inflammatory disorder or disease.

17. Process for controlling an urological disorder or disease in humans and animals by administration of a BETA 3-agonistically effective amount of at least one compound according to claim 1.

18. Process for controlling an inflammatory disorder or disease in humans and animals by administration of a BETA 3-agonistically effective amount of at least one compound according to claim 1.
